(19) United States
(12) Patent Application Publication
(10) Pub. No.: US 2005/0129769 A1
(43) Pub. Date: Jun. 16, 2005

(54) Polymeric articles for carrying therapeutic agents

(76) Inventors: Stephen E. Barry, Alamo, CA (US); Andrew A. Goodwin, San Leandro, CA (US); Rachel Decor, Berkeley, CA (US)

Correspondence Address: JACQUELINE S LARSON, P O BOX 2426, SANTA CLARA, CA, 95055-2426, US

(21) Appl. No.: 10/995,880

(22) Filed: Nov. 22, 2004

Related U.S. Application Data
(63) Continuation-in-part of application No. PCT/US03/17536, filed on Jun. 3, 2003.
(60) Provisional application No. 60/385,537, filed on Jun. 3, 2002.

(51) Int. Cl. C08G 63/48; C08G 63/91; A61K 9/14; C08B 33/02; C07C 323/25

(52) U.S. Cl. 424/486; 525/54.2; 536/110; 562/557

(57) ABSTRACT

Hydrophilic polymeric nanoarticles comprising a polymeric scaffold and one or more therapeutic agents, such as drug or drug-conjugate molecules, covalently attached to the scaffold. The articles may further optionally comprise recognition elements (REs) that bind to biomolecular structures expressed on certain cells or in certain tissues, to facilitate targeting and/or delivery.

The present invention is a continuation-in-part of co-pending International Patent Appln. No. PCT/US03/17536, filed Jun. 3, 2003 and designating the United States of America, which application claims the benefit of Provisional U.S. patent application Ser. No. 60/385,537, filed Jun. 3, 2002; the entire disclosures of both of which are incorporated herein by reference.

### FIELD OF THE INVENTION

The present invention is directed to the field of therapeutic entities. More specifically, this invention relates to hydrophilic polymeric articles incorporating therapeutic components, such as drugs.

### BACKGROUND OF THE INVENTION

Small molecule drugs are often comprised of substantial hydrophobic structures that have limited water and blood solubility. These compounds will interact strongly with biomembranes, will rapidly diffuse through biomembranes, and thus will readily exit the bloodstream and nonspecifically penetrate tissues. Such undesirable distribution can lead to high dosing requirements, rapid clearance, and toxic side effects. In fact, small molecule drugs can pose toxicity challenges regardless of hydrophobic or hydrophilic nature. To improve performance, drugs have been covalently conjugated to water-soluble, synthetic polymers, such as N-(2-hydroxypropyl)methacrylamide (HPMA) (Burtles et al., Hum. Exp. Toxicol., 1998, 17, 93-104); natural polymers, such as dextran; and proteins, such as albumin (Schutte M. T., et al., Crit. Rev. Ther. Drug Carrier Syst., 1999, 16, 245-288); for purposes such as altering biodistribution, reducing toxicity, reducing multi drug resistance (MDR) efflux, and prolonging activity. Size and solubility provide a basis for understanding the differences between the drug compared to the drug conjugate. Attachment to hydrophilic polymers produces a larger molecule with decreased diffusivity and increased water and blood solubility. Nonspecific membrane penetration is thus substantially reduced. Such drug-polymer conjugates thus reside in the bloodstream longer in comparison to the drug alone and may therefore be passively targeted to tissue that has a leaky vasculature.

Active targeting of drugs has been achieved through the use of ligands constructs capable of binding to specific biomolecular structures, such as receptors overexpressed on certain cancer cells, for the purpose of localizing the bioactive agents to a desired type of cell or cellular component, type of tissue or tissue component, or organ. For example, the HPMA-drug conjugates have been further functionalized with monoclonal antibodies that target receptors overexpressed on certain cancers. In another example, drug molecules have been directly conjugated to monoclonal antibodies (mAb). For instance, Seattle Genetics (Seattle, Wash.) has a compound, SGN 15, which is a mAb that is functionalized with doxorubicin (dox) and binds to surface Ley antigens expressed in certain tumors.

Other materials and methods that improve performance, for example through improved therapeutic efficacy, larger therapeutic index, improved solubility, or lower dosage requirement beyond the current state of the art would be important additions to the current art.

### SUMMARY OF THE INVENTION

As used herein, the terms “articles” and “nanoarticles” are used interchangeably.

This invention is directed to hydrophilic polymeric nanoarticles comprising i) a scaffold comprised of crosslinked hydrophilic building blocks and ii) one or more therapeutic agents, such as drug or drug-conjugate molecules, covalently attached to the scaffold. The nanoarticles may further optionally comprise one or more recognition elements (REs) to facilitate targeting and/or delivery. The nanoarticles may also optionally be comprised of polyethylene glycol (PEG)-based molecules. The PEG chains may serve as linkers or tethers, with one end attached to the nanoarticle surface and the other end functionalized with an RE, and/or the PEG chains may provide other useful or desirable characteristics to the hydrophilic nanoarticles. The invention is further directed to methods of synthesizing these nanoarticles, to novel materials utilized in the synthesis of the nanoarticles, and to the various applications for which the nanoarticles may be used.

The “nanoarticles” of the invention are less than 1000 nm in diameter. They are preferably from about 5 nm to about 100 nm in diameter, more preferably from about 10 nm to about 70 nm. The size of the nanoarticles allows their use as bioactive entities in mammals. To avoid uptake by the reticuloendothelial system, nanoarticles are preferably less than 100 nm. To avoid renal clearance, nanoarticles are preferably larger than 5 nm. Reverse microemulsion polymerization can yield nanoarticle scaffolds of the invention.

Therapeutic agents are incorporated into the nanoarticles through covalent linkage to the hydrogel scaffold. A preferred therapeutic entity is doxorubicin or doxorubicin derivatives.

REs serve to bind the nanoarticle of the invention to desired biomolecules overexpressed or otherwise found on certain cell surfaces or in certain tissues. The number of REs per nanoarticle can range from 2 to about 1000, preferably from 2 to 500. The nanoarticles may optionally further be comprised of more than one type of RE. As used herein, a RE “type” is defined as an RE of a specific molecular structure. An additional advantage of the present invention is that multiple RE types with complementary features may be incorporated into a single nanoarticle.

The nanoarticle scaffolds are comprised of crosslinked hydrophilic building blocks. The building blocks are crosslinked in the dispersed aqueous phase of a reverse microemulsion. Carbohydrates and carbohydrate derivatives are preferably used as building blocks.

### DETAILED DESCRIPTION OF THE INVENTION

When used herein and in the appended claims, the terms “a” and “an” mean “one or more”, unless otherwise indicated.

By “water-soluble” is meant, herein and in the appended claims, having a solubility in water of greater that 10 mg/mL, and preferably greater than 50 mg/mL.

The nanoarticles of this invention are comprised of two types of molecular structures: scaffolds and therapeutic agents. In a presently preferred embodiment, the nanoarticles also comprise a third molecular structure: REs that bind to biomolecules expressed on certain cells or in certain tissues. The REs and the therapeutic agents are covalently attached to the nanoarticle scaffold. As used herein, the terms “nanoarticle scaffold”, “hydrogel scaffold” and “scaffold” are used interchangeably and refer to the portion of the nanoarticle (the polymeric matrix structure) that is formed prior to attachment of bioactive agents and recognition elements. The scaffold of the present invention is a chemically crosslinked, nanoscopic hydrogel structure.

The nanoarticles are hydrophilic and intended for use in mammals. In one embodiment, each nanoarticle is functionalized with two or more recognition elements that possess high affinity to biomolecular targets, the recognition elements being covalently linked to the nanoarticle polymeric matrix structure. The invention is further directed to methods of synthesizing the polymeric nanoarticles.

The nanoarticles of the invention may range in size from about 5 nm to about 1000 nm, more preferably from about 5 nm to about 100 nm in diameter, and most preferably about 10 nm to about 70 nm. Nanoarticles in the 10 to 70 nm size range may effectively avoid renal clearance and uptake by the reticuloendothelial system. Additionally, such nanoarticles may advantageously exit the blood stream to reach desired cell, tissue, or organ targets.

The nanoarticles of the present invention may, in particular, be advantageously used in the treatment of cancer. The leaky vasculature found in tumors may allow these nanoarticles to leave the blood stream and concentrate in tumors. This effect, described as enhanced permeability and retention (EPR) for macromolecular agents, has been observed to be universal in solid tumors (Maeda H., et al., J. Controlled Release, 2000, 65, p. 271-284). The key mechanism for the EPR effect for macromolecules is retention, whereas low-molecular-weight substances are not retained but are returned to circulating blood by diffusion. Nanoarticles of diameters from 5 to 100 nm can thus accumulate in solid tumors. Thus, the nanoarticles of the present invention will naturally concentrate in tumors even prior to target binding, providing for greater efficacy and less systemic toxicity.

Hydrophilic building blocks with polymerizable groups are employed to form the hydrogel scaffold. The building blocks are crosslinked in the dispersed aqueous phase of reverse microemulsions. The number of polymerizable groups attached to one single building block can range, for example, from about one to three for low molecular weight building blocks, to ten or more for polymeric building blocks. Building blocks that contain more than one polymerizable group can act as crosslinking agents and enable the formation of a hydrogel scaffold. Using different amounts and proportions of building blocks from a set of building blocks with one, two, or more polymerizable groups allows formation of hydrogels of different compliancy upon polymerization.

Exemplary polymerizable groups include, but are not limited to, acrylate, acrylamide, methacrylate, methacrylamide, vinyl ether, styryl, epoxide, maleic acid derivative, diene, substituted diene, thiol, alcohol, amine, hydroxyamine, carboxylic acid, carboxylic anhydride, carboxylic acid halide, aldehyde, ketone, isocyanate, succinimide, carboxylic acid hydrazide, glycidyl ether, siloxane, alkoxysilane, alkyne, azide, 2′-pyridyldithiol, phenylglyoxal, iodo, maleimide, imidoester, dibromopropionate, and iodacetyl moieties.

Free-Radical Polymerization: Preferred polymerizable functionalities are acrylate, acrylamide, methacrylate, and methacrylamide moieties. Such moieties are amenable to free-radical polymerization. Free-radical polymerization can be readily achieved through the combination of UV light and photoinitiators, redox-coupled free-radical initiators, or heat and heat-activated initiators.

Building blocks that may be used to form the article scaffold include small molecules with one polymerizable group or multiple polymerizable moieties that can act as small molecule crosslinkers. Exemplary building blocks include acrylamide, sodium acrylate (NaA), diacetone acrylamide (DAA), malonate acrylamide (MalAc), levulinic acrylamide, methylene bisacrylamide, ammonium 2,2-bisacrylamidoacetate, 2-acrylamidoglycolic acid, 2-aminoethyl methacrylate, N-(3-aminopropyl)methacrylamide (APMA), ornithine mono-acrylamide, ornithine diacrylamide sodium salt, N-acryloyltris(hydroxymethyl)-methylamine, hydroxyethylacrylate, N-(2-hydroxypropyl)methacrylamide, N-(2-hydroxypropyl)acrylamide, 2-sulfoethylmeth-acrylate, 2-methacryloylethyl glucoside, glucose monoacrylate, glucose-1-(N-methyl)acrylamide, glucose-2-acrylamide, glucose-1,2-diacrylamide, maltose-1-acrylamide, sorbitol monoacrylate, sorbitol diacrylate, sucrose diacrylate, sucrose mono(ethylenediamine acrylamide), sucrose di(ethylenediamine acrylamide), sucrose di(diethylenetriamine acrylamide), kanamycin tetraacrylamide, kanamycin diacrylamide, sucrose mono(ethylenediamine acrylamide) mono(diethylenetriamine acrylamide) mono(phenyl alanine) sodium salt, as well as other acrylate- or acrylamide-derivatized sugars.

Building blocks are chosen to achieve a desired content of certain functionalities in the article scaffold. Such functionalities can improve solubility and may also be used as points of attachment for therapeutic agents and REs. For instance, APMA may be used to introduce amines, sodium acrylate may be used to introduce carboxylates, and DAA may be used to incorporate ketones. In a preferred embodiment, at least some of the building blocks are a N,N′-cystinebisacrylamide (CiBA) monomer, which has the following formula I:

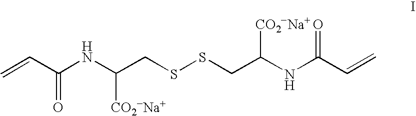

CIBA may be prepared by reacting L-cystine (II) with 2 equivalents of acryloyl chloride (III), according to the following reaction scheme:

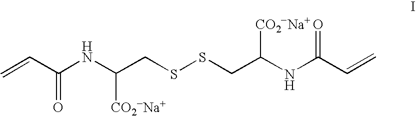

CiBA is water-soluble and is capable of polymerizing with other scaffold building blocks, such as acrylate-functionalized carbohydrates, to form the hydrogel scaffold. In addition, its disulfide linkage provides, after reduction, free thiols for linker attachment.

In a preferred embodiment, at least some of the building blocks are carbohydrates. In the case of carbohydrate building blocks, the carbohydrate region is comprised of a plurality of hydroxyl groups, wherein at least one hydroxyl group is modified to include at least one polymerizable group.

The carbohydrate region of the carbohydrate building block may include a carbohydrate or carbohydrate derivative. For example, the carbohydrate region may be derived from a simple sugar, such as N-acetylglucosamine, N-acetylgalctosamine, N-acetylneuraminic acid, neuraminic acid, galacturonic acid, glucuronic acid, ioduronic acid, glucose, ribose, arabinose, xylose, lyxose, allose, altrose, apiose, mannose, gulose, idose, galactose, fucose, fructose, fructofuranose, rhamnose, arabinofuranose, and talose; a disaccharide, such as maltose, sucrose, lactose, or trehalose; a trisaccharide; a polysaccharide, such as cellulose, starch, glycogen, alginates, inulin, pullulan, dextran, dextran sulfate, chitosan, glycosaminoglycans, heparin, heparin sulfate, hyaluronates, tragacanth gums, xanthan, other carboxylic acid-containing carbohydrates, uronic acid-containing carbohydrates, lactulose, arabinogalactan, and their derivatives, and mixtures of any of these; or modified polysaccharides. Other representative carbohydrates include sorbitan, sorbitol, chitosan and glucosamine. The carbohydrate may include amine groups in addition to hydroxyl groups, and the amine or hydroxyl groups can be modified, or replaced, to include a crosslinking group, other functionalities, or combinations thereof.

Carbohydrate-based building blocks may be prepared from the carbohydrate precursor (e.g. sucrose, inulin, dextran, pullulan, etc.) by coupling technologies known in the art of bioorganic chemistry (see, for example, G Hermanson, Bioconjugation Techniques, Academic Press, San Diego, 1996, pp 27-40,155,183-185, 615-617; and S. Hanesian, Preparative Carbohydrate Chemistry, Marcel Dekker, New York, 1997.) For example, a crosslinkable group can be attached to a carbohydrate via the dropwise addition of acryloyl chloride to an amine-functionalized sugar. Amine-functionalized sugars can be prepared by the reaction of ethylene diamine (or other amines) with 1,1′-carbonyldiimidazole-activated sugars. Ester-linked reactive groups can be synthesized through the reaction of acrylic or methacrylic anhydrides with the hydroxyl group of a carbohydrate such as inulin in pyridine.

Carbohydrate-based building blocks may also be prepared by the partial (or complete) functionalization of the carbohydrate with moieties that are known to polymerize under free radical conditions. For example, methacrylic esters may be placed on a carbohydrate at varying substitution levels by the reaction of the carbohydrate with methacrylic anhydride or glycidyl methacrylate (Vervoort L., et al., International Journal of Pharmaceutics, 1998, 172, 127-135).

In a presently preferred embodiment, at least some of the building blocks are inulin multi-methacrylate (IMMA) monomer, which has the following formula IV: 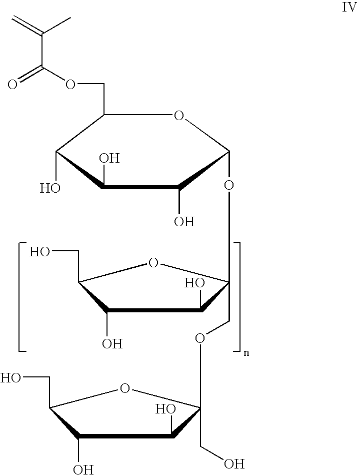
The value of n in formula IV is from about 5 to about 50. In a presently preferred embodiment, inulin with an average degree of polymerization (DOP) of about 10 to about 20 is used. Thus, in a preferred embodiment, n is from about 8 to about 18. The extent to which inulin is functionalized with methacrylate moieties, that is, the number of hydroxyl moieties on inulin that are converted to methacrylic esters to produce IMMA, is a statistical process governed by the concentrations and weight ratios of inulin and methacrylic anhydride starting material. The extent of functionalization may range from one methacrylate for every 1 to 100 monosaccharide repeat units, more preferably one methacrylate for every 3 to 20 monosaccharide repeat units. The ester linkage to inulin may advantageously function as a site of degradation in vivo, allowing the article to degrade and be cleared from the body. Dextran multimethacrylamide and pullulan multimethacrylamide are additional preferred building blocks that may be prepared using similar methods.

Carbohydrate-based building blocks may also be prepared by chemoenzymatic methods (Martin B. D., et al., Macromolecules, 1992, 25, 7081), for example in which Pseudomonas cepacia catalyzes the transesterification of monosaccharides with vinyl acrylate in pyridine or by the direct addition of an acrylate (Piletsky S., et al., Macromolecules, 1999, 32, 633-636). Other functional groups may be present, as numerous derivatized carbohydrates are known to those familiar with the art of carbohydrate chemistry.

The carbohydrate structures are chosen in part for their hydrophilicity. Nanoarticles that incorporate substantially hydrophobic drugs must possess highly hydrophilic scaffolds in order that high water solubility be maintained after functionalization with the drug. The nanoarticles of the present invention that are comprised of IMMA have a high water content. “High water content”, as used herein and the appended claims, means an article comprised of about 65 to about 98 wt % water, more preferably about 75 to about 98 wt % water, and most preferably about 80 to 97 wt % water. Thus, the amount of breakdown products is less than articles with a higher polymer concentration. The high water content scaffolds also can reduce immunogenicity, because there are fewer surfaces for immune system components to interact with.

Besides carbohydrate-based building blocks, other examples of acrylate- or acrylamide-derivatized polymeric building blocks include polyethylene glycol-based molecules, such as polyethyleneglycol diacrylate, with molecular weights ranging from 200 to 40,000 daltons.

In a preferred embodiment, to facilitate metabolism of the hydrogel scaffold and thereby drug release in a desired time frame, degradable linkages are included within the crosslinked scaffold. Degradable linkages can be included through the use of polylactide, polyglycolide, poly(lactide-co-glycolide), polyphosphazine, polyposphate, polycarbonate, polyamino acid, polyanhydride, and polyorthoester-based building blocks, among others. Additionally, degradable linkages may be used to attach polymerizable moieties to carbohydrates. For instance, IMMA contains ester moieties that connect the inulin carbohydrate backbone to the alkyl chain that is formed upon free radical polymerization used to generate the scaffold of the present invention. Additionally, small molecule crosslinking agents containing similar hydrolyzable moieties as the polymers such as carbonates, esters, urethanes, orthoesters, amides, and phosphates may be used as building blocks. To function as degradable components in the hydrogel scaffold, these building blocks must be functionalized with two or more polymerizable moieties. For example, polyglycolide diacrylate, polyorthoester diacrylate and acrylate-substituted polyphosphazine, acrylate-substituted polyamino acid, or acrylate-substituted polyphosphate polymers can be used as degradable building blocks. Methacrylate or acrylamide moieties can be employed instead of acrylate moieties in the above examples. Similarly, small molecules containing a hydrolyzable segment and two or more acrylates, methacrylates, or acrylamides may be used. Such degradable polymers and small molecule building blocks may be functionalized with acrylate, methacrylate, acrylamide or similar moieties by methods known in the art.

The nanoarticle scaffolds and the scaffold breakdown products of this invention are designed to be non-toxic and eliminated from the body. They may have degradable, preferably carbohydrate-based, polyamino acid-based, polyester-based, or PEG-based cores, with the rate of degradation controlled by the identity of the sugar, crosslink density, and other features. Thus, the articles can be metabolized in the body, preventing undesirable accumulation in the body.

Chemoselective Polymerizations: Chemoselective building blocks may also be used to form the scaffold. A representative example of this strategy may be the use of a polysaccharide that has been partially oxidized to contain numerous aldehydes within a reverse microemulsion. A di(amino-oxy) containing compound, such as that made from reacting ethylene diamine with the NHS ester of Boc-amino-oxyacetic acid (see the following Reaction Scheme), can then be used as a crosslinking agent through the reaction of the aldehydes of the oxidized sugar reacting with the amino-oxy functionalities.

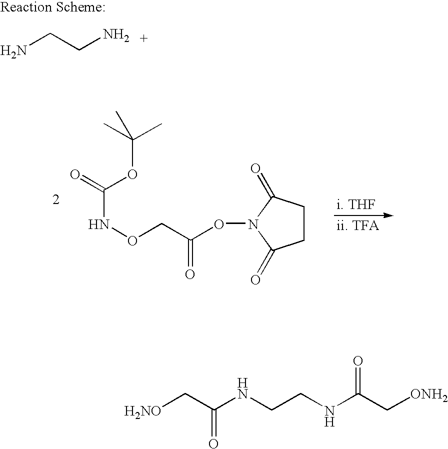

Article Scaffold Fabrication in Reverse Microemulsions: Articles of the present invention are fabricated by first forming nanoscopic hydrogel scaffolds through the crosslinking of hydrophilic building blocks solubilized in the dispersed water phase of a reverse microemulsion. The organic solvent and non-reactive surfactants are removed after polymerization to yield crosslinked, water-soluble nanoscopic articles.

Reverse microemulsions for scaffold fabrication are formed by combining aqueous buffer or water, building blocks, organic solvent, surfactants and initiators in the appropriate ratios to yield a stable phase of surfactant-stabilized aqueous nanodroplets dispersed in a continuous oil phase. Stable reverse microemulsion formulations can be found using known methods by those skilled in the art. They are discussed, for example, in Microemulsion Systems, edited by H. L. Rosano and M. Clausse, New York, N.Y.: M. Dekker, 1987; and in Handbook of Microemulsion Science and Technology, edited by P. Kumar and K. L. Mittel, New York, N.Y.: M. Dekker, 1999. In this invention, an aqueous phase with solubilized hydrophilic building blocks is added to an organic solvent containing one or more solubilized surfactants to form a reverse microemulsion.

The dispersed aqueous phase contains hydrophilic building blocks solubilized at about 5 to about 65 wt %, preferably about 5 to about 25 wt %, most preferably 10 to 20 wt %. While not wishing to be bound by theory, the use of high water-content hydrogel scaffolds also may reduce immunogenicity in end uses, because there is less foreign surface for immune system components to recognize. The high water content also provides compliancy through a more flexible scaffold. Thus, when attaching to cell surface receptors, the articles are able to conform to the cell surface, allowing more surface receptors to be bound. Binding more receptors may allow the article to better function as an antagonist. Additionally, while not wishing to be bound by theory, it is believed that article cell surface coverage can inhibit other cell signaling pathways.

Polymerization of the building blocks in the nanodroplets of the dispersed aqueous phase of the reverse microemulsion follows procedures known to those skilled in the art (see, for example, Odian G. G.; Principles of Polymerization, 3^{rd} Ed., Wiley, New York, 1991; L. H. Sperling, Introduction to Physical Polymer Science, Chapter 1, pp. 1-21, John Wiley and Sons, New York, 1986; and R. B. Seymour and C. E. Carraher, Polymer Chemistry, Chapters 7-11, pp. 193-356, Dekker, New York, 1981). Polymerization has been performed in the dispersed phase of microemulsions and reverse microemulsions (for a review, see Antonietti, M.; and Basten, R., Macromol. Chem. Phys. 1995, 196, 441; for a study of the polymerization of a hydrophilic monomer in the dispersed aqueous phase of a reverse microemulsion, see Holtzscherer, C.; and Candau, F., Colloids and Surfaces, 1988, 29, 411). Such polymerization may yield articles in the 5 nm to 50 nm size range.

The size of the nanodroplets of the dispersed aqueous phase is determined by the relative amounts of water, surfactant and oil phases employed. Surfactants are utilized to stabilize the reverse microemulsion. These surfactants do not include crosslinkable moieties; they are not building blocks. Surfactants that may be used include commercially available surfactants such as Aerosol OT (AOT), polyethyleneoxy(n)nonylphenol (Igepal™, Rhodia Inc. Surfactants and Specialties, Cranbrook, N.J.), sorbitan esters including sorbitan monooleate (Span® 80), sorbitan monolaurate (Span® 20), sorbitan monopalmitate (Span® 40), sorbitan monostearate (Span® 60), sorbitan trioleate (Span® 85), and sorbitan tristearate (Span® 65), which are available, for example, from Sigma (St Louis, Mo.). Sorbitan sesquioleate (Span® 83) is available from Aldrich Chemical Co., Inc. (Milwaukee, Wis.). Other surfactants that may be used include polyoxyethylenesorbitan (Tween®) compounds. Exemplary cosurfactants include polyoxyethylenesorbitan monolaurate (Tween® 20 and Tween® 21), polyoxyethylenesorbitan monooleate (Tween® 80 and Tween® 80R), polyoxyethylenesorbitan monopalmitate (Tween® 40), polyoxyethylenesorbitan monostearate (Tween® 60 and Tween® 61), polyoxyethylenesorbitan trioleate (Tween® 85), and polyoxyethylenesorbitan tristearate (Tween® 65), which are available, for example, from Sigma (St Louis, Mo.). Other exemplary commercially available surfactants include polyethyleneoxy(40)-sorbitol hexaoleate ester (Atlas G-1086, ICI Specialties, Wilmington Del.), hexadecyltrimethylammonium bromide (CTAB, Aldrich), and linear alkylbenzene sulfonates (LAS, Ashland Chemical Co., Columbus, Ohio).

Other exemplary surfactants include fatty acid soaps, alkyl phosphates and dialkylphosphates, alkyl sulfates, alkyl sulfonates, primary amine salts, secondary amine salts, tertiary amine salts, quaternary amine salts, n-alkyl xanthates, n-alkyl ethoxylated sulfates, dialkyl sulfosuccinate salts, n-alkyl dimethyl betaines, n-alkyl phenyl polyoxyethylene ethers, n-alkyl polyoxyethylene ethers, sorbitan esters, polyethyleneoxy sorbitan esters, sorbitol esters and polyethyleneoxy sorbitol esters.

Other surfactants include lipids, such as phospholipids, glycolipids, cholesterol and cholesterol derivatives. Exemplary lipids include fatty acids or molecules comprising fatty acids, wherein the fatty acids include, for example, palmitate, oleate, laurate, myristate, stearate, arachidate, behenate, lignocerate, palmitoleate, linoleate, linolenate, and arachidonate, and salts thereof such as sodium salts. The fatty acids may be modified, for example, by conversion of the acid functionality to a sulfonate by a coupling reaction to a small molecule containing that moiety, or by other functional group conversions known to those skilled in the art.

Additionally, polyvinyl alcohol (PVA), polyvinylpirolidone (PVP), starch and their derivatives may find use as surfactants in the present invention.

Cationic lipids may be used as cosurfactants, such as cetyl trimethylammonium bromide/chloride (CTAB/CTAC), dioctadecyl dimethyl ammonium bromide/chloride (DODAB/DODAC), 1,2-diacyl-3-trimethylammonium propane (DOTAP), 1,2-diacyl-3-dimethyl ammonium propane (DODAP), [2,3-bis(oleoyl)propyl]trimethyl ammonium chloride (DOTMA), and [N-(N′-dimethylaminoethane)-carbamoyl]cholesterol, dioleoyl) (DC-Chol). Alcohols may also be used as cosurfactants, such as propanol, butanol, pentanol, hexanol, heptanol and octanol. Other alcohols with longer carbon chains may also be used.

Incorporation of Drug Molecules into Articles: The terms “drug”, “drug-conjugate”, “bioactive agent” and “therapeutic agent” are used interchangeably herein. In a preferred embodiment, drug molecules are covalently linked to the article scaffold. For example, the chemotherapeutic doxorubicin may be attached to the scaffold through a EDC coupling reaction between the amine moiety on doxorubicin and a carboxylic acid moiety included in the hydrogel scaffold, for example by using sodium acrylate (NaA), malonate acrylamide (MalAc) or CiBA as a building block. In another embodiment, doxorubicin may be attached via an imine bond by reacting doxorubicin's amine moiety with an aldehyde moiety of the hydrogel scaffold. An aldehyde may be created by first using a carbohydrate-based building block to form the article, and then oxidizing the carbohydrate after the article is formed. In another embodiment, doxorubicin may be attached to the article matrix through its ketone moiety. Carbohydrazide or other dihydrazide or di-amino-oxy functionalized structures may be used to link doxorubicin to a scaffold that contains an aldehyde or ketone through the formation of a hydrazone bond. An aldehyde or ketone may be incorporated into the scaffold through the use of a ketone-containing acrylate building block such as DAA. A hydrazone bond may favorably release the therapeutic compound under the mildly acidic physiological conditions encountered upon article endocytosis and entrance into lysosomes.

In another embodiment, nanoarticle scaffolds comprised of amino groups, for example through the inclusion of APMA or methacrylate-functionalized short peptide (prepared according to U.S. Pat. No. 5,037,883) building blocks, may be used to covalently attach cyclosporins that contain carboxylate linkages. Cyclosporin drugs may find applications for pathologies that benefit from immunosuppression, such as inflammatory diseases, and for organ transplantation.

In another embodiment, nanoarticle scaffolds comprised of aldehyde or ketone groups (for example incorporated through the inclusion of DAA, levulinic acrylamide or oxidized carboxylates such as inulin or dextran building blocks) may be used to covalently attach drugs or drug derivatives that contain a moiety, for instance calicheamicin, through the use of a hydrazone coupling scheme. This coupling scheme results in a hydrazone bond that is hydrolytically labile, especially at low pH found in lysosomes (Bernstein I., et al., Bioconjugate Chem., 2002, 13, 40-46).

In another embodiment, nanoarticle scaffolds comprised of acid or anhydride groups, for example incorporated through the inclusion of sodium acrylate or anhydride building blocks, may be used to covalently attach dexamethasone, through the use of an amide coupling scheme.

Nanoarticle scaffolds comprised of carboxylate groups (for example, incorporated through the inclusion of sodium acrylate (NaA), CiBA or MAlAc building blocks) may be used to covalently attach drugs or drug derivatives that contain an amine moiety, for instance peptide-modified camptothecin (Frigerio E., et al., J. Controlled Release, 2000, 65, 105-119) through an EDC-NHS coupling scheme.

In another embodiment, when the nanoarticle scaffolds are comprised of aldehyde or ketone groups (which may be incorporated through the use of DM, levulinic acrylamide or oxidized carboxylates such as inulin or dextran), a drug or drug derivative possessing an amine, such as gemcitabine, may be incorporated through the use of a “Schiff base” coupling scheme. The imide bond formed from the attachment of gemcitabine to DAA can be cleaved in acidic media. During internalization, the drug is taken up by the cell, where it is exposed to the acidic environment of the lysosome, thereby releasing gemcitabine in its unmodified form.

In another embodiment, nanoarticle scaffolds comprised of carboxyl groups (for example, incorporated through the inclusion of CiBA, MalAc or NaA building blocks) may be used to covalently attach drugs or drug derivatives that contain a moiety, for instance salicylic acid, through the use of an EDC-NHS coupling scheme. For instance, the hydroxyl group of salicylic acid will react with the carboxyl group of the CiBA, MalAc or NaA to form an ester link. Hydrolysis or the enzyme esterase will cleave the ester bond between salicylic acid and the carboxylic acid groups of CiBA or NaA, releasing salicylic acid in an unmodified form.

In another embodiment, a drug structure may be modified to facilitate attachment to a nanoarticle scaffold. For instance, the 2′ hydroxyl group of paclitaxel can be reacted with multiple linkers that enable the coupling to nanoarticle scaffolds. For example, the acid moiety of a resin-immobilized glycine linker can be attached to paclitaxel using a carbodiimide; the resulting compound can be cleaved at the site of the amine using 1% TFA, producing a free amine which can be conjugated with nanoarticles possessing carboxylates using an EDC coupling scheme.

In another embodiment, paclitaxel-2′-succinate (Deutsch H., et al., J. Med. Chem., 1989, 32, 788-792) conjugation to the nanoarticle is possible using a carbodiimide-mediated amide coupling. This coupling occurs between the paclitaxel-2′-succinate group and an amine group of the APMA component of the nanoarticle to form a labile ester.

In another embodiment, the nanoarticle can be directly coupled to paclitaxel by reacting the acid-functionalized (NaA) nanoarticle to the 2′-hydroxyl group of paclitaxel. This chemical pathway has been previously described using a poly(L-glutamic acid)-paclitaxel conjugate (Li H., et al., Cancer Res., 1998, 58, 2404-2409).

In another embodiment, nanoarticle scaffolds containing carboxylic acids, for example incorporated through the inclusion of sodium acrylate (NaA) building blocks, may be used to covalently attach drugs or drug derivatives that contain a moiety, for instance 5-flourouracil (5FU) (or derivatives) through the use of an amide forming coupling reaction between an amine-functionalized 5FU derivative and the carboxylic acids located on the nano-article. The synthesis of 1-alkylcarbonyloxymethyl derivatives of 5FU has been previously described and those materials have been demonstrated to release 5FU in an unmodified form (Taylor H. E.; Sloan K. B., Journal of Pharmaceutical Sciences, 1998, 87, 15). The application of this synthetic route will yield the necessary amine-functionalized 5FU, whilst realizing a similar release profile.

Nanoarticle scaffolds comprised of acid groups (for example incorporated through the inclusion of CiBA, MalAc and NaA building blocks) may be used to covalently attach drugs or drug derivatives that contain a carboxylate moiety, for instance methotrexate, by first coupling the drug or drug derivative to boc-protected ethanolamine to form an ester, and then coupling to the nanoarticle through an EDC coupling scheme after deprotecting the modified drug. This ester conjugate is known to hydrolyze at low pH, releasing the drug in its original form (Wilson J. M., et al., Biochem Biophys. Res. Commun., 1992, 184, 300-305; Ohkuma S., Poole B., Proc. Natl. Acad. Sci. USA, 1978, 75, 3327-3331). Such conditions of low pH are found in cellular lysosomes. These nanoarticles may find use in the treatment of multiple pathologies, including cancer and inflammatory conditions such as rheumatoid arthritis and inflammatory bowel disease.

In another embodiment, platinum conjugates are incorporated onto nanoarticle scaffolds. The platinum may be in the II^{nd} or IV^{th} oxidation state. There remains a distinct need for new platinum chelates with further improvements in therapeutic index compared with the currently-approved platinum chelates. Such chelates should be highly water-soluble and stable in an aqueous environment, but sufficiently labile in tumor cells to provide species capable of crosslinking DNA and ultimately causing tumor cell death. The hydrogel network of the nanoarticles of the present invention combined with the capacity to fine tune its chemical composition allows for such a high water solubility and flexibility in the way to attach chelates, which constitute a significant improvement over WO 9847537. It has been shown that changes in the platinum chelate structure could modify the spectrum of tumor types for which platinum therapy is effective and/or alter the toxicity profile of the chelate. In one important embodiment of the invention the platinum is complexed to the hydrogel matrix via O,N-ligation, which is expected to yield a more stable compound.

This can be accomplished preferentially for nanoarticles obtained by free-radical polymerization, and containing a combination of acid functions (such as from NaA) and amines (such as from APMA) or amides moieties (such as acrylamide), or building blocks carrying both types of functions such as CIBA, MalAc or methacryloylate-functionalized short peptides made according to U.S. Pat. No. 5,037,883. Such moieties provide attachment points to generate a O,N-cis platinum nanoarticle conjugate, leaving open the possibility of targeting the nanoarticle.

Finally, the high toxicity of platinum derivatives renders the specific targeting of the nanoarticles to organs or tumor sites of paramount benefit to increase the therapeutic index.

Drugs that may find use in the present invention include those that act on the peripheral nerves, adrenergic receptors, cholinergic receptors, nervous system, skeletal muscles, cardiovascular system, smooth muscles, blood circulatory system, synaptic sites, neuro-effector junctional sites, endocrine system, hormone systems, immunological system, reproductive system, skeletal system, autocoid systems, alimentary and excretory systems, histamine systems, respiratory system, reticuloendothelial system, skeletal system, skeletal muscles, smooth muscles, immunological system, reproductive system, cancerous tissues, and the like. The active drug that can be delivered for acting on these recipients includes, but is not limited to, anticonvulsants, analgesics, anti-parkinsons, anti-inflammatories, calcium antagonists, anesthetics, antimicrobials, antimalarials, antiparasitics, antihypertensives, antihistamines, antipyretics, alpha-adrenergic agonists, alpha-blockers, biocides, bactericides, bronchial dilators, beta-adrenergic blocking drugs, contraceptives, chemotherpeutics, cardiovascular drugs, calcium channel inhibitors, depressants, diagnostics, diuretics, electrolytes, enzymes, hypnotics, hormones, hypoglycemics, hyperglycemics, muscle contractants, muscle relaxants, neoplastics, glycoproteins, nucleoproteins, lipoproteins, ophthalmics, psychic energizers, sedatives, steroids, sympathomimetics, parasympathomimetics, tranquilizers, urinary tract drugs, vaccines, vaginal drugs, vitamins, nonsteroidal anti-inflammatory drugs, angiotensin converting enzymes, polynucleotides, polypeptides, polysaccharides, and the like.

In a presently preferred embodiment, drugs that may be advantageously employed in the present invention include, but are not limited to, chemotherapeutics such as doxorubicin, paclitaxel, gemcitibine, vincristine, cisplatin, carboplatin, chlorambucil, topotecan, methotrexate, derivatives of these compounds, and any other FDA-approved chemotherapeutic, as well as molecules that may act as chemotherapeutics but that are not yet commercialized, and derivatives and analogues of all of the above chemotherapeutics.

The therapeutic agent for delivery in this invention can be in various pharmaceutically acceptable forms, such as prodrugs, uncharged molecules, molecular complexes, and pharmacologically acceptable salts. Derivatives of medicines, such as esters, ethers and amides, can be used.

Article Functionalization with Recognition Elements: After the assembled building blocks are crosslinked to form the hydrogel scaffold and the therapeutic agent has been covalently attached to the scaffold, the article surface may be functionalized with REs. The REs can be linked either directly or through a linker molecule to the surface of the nanoparticle. In a linker configuration, part or all of the REs are “displayed” at the end terminus of the tether. Therefore, in one application of the invention, the articles consist of REs displayed on a hydrogel scaffold. In another embodiment of the patent, the articles consist of an RE, such as a high affinity peptide, linked to the surface of the article core scaffold via a linker molecule, the linker comprising preferentially polyethylene glycol (PEG).

For each of these embodiments, it is possible to functionalize the articles with several coupling strategies, varying both the order of addition of the different components and the reactive chemical moieties used for the coupling.

The components may be attached to one another in the following sequences. The hydrogel scaffold is first reacted with a di-functional PEG-containing tether, followed by functionalization of the free terminus of a portion of the PEG chain with a RE. Alternatively, the RE is coupled first to the PEG-containing tether, followed by the attachment of the other PEG terminus to the scaffold.

Several combinations of reactive moieties can be chosen to attach the RE to the tether and to react the tether with the scaffold. In using a series of orthogonal reaction sets, varying some of the scaffold building blocks and/or tethering arms, it is also possible to attach REs with different molecular structures that bind to different receptors, onto the same article scaffold in well-controlled proportions. Reactions using orthogonal reactive pairs can be done simultaneously or sequentially.

As far as reaction conditions are concerned, it is preferable to functionalize the articles in an aqueous system. The surfactants and the oil phase, residual from the synthesis of the hydrogel scaffold, can be removed through the use (singularly or in combination) of solvent washing, for instance using ethanol to solubilize the surfactant and oil while precipitating the articles; surfactant-adsorbing beads; dialysis; or the use of aqueous systems such as 4M urea. Methods for surfactant removal are known in the art.

The RE must contain a functionality that allows its attachment to the article. Preferentially, although not necessarily, this functionality is one member of a pair of chemoselective reagents selected to aid the coupling reaction (Lemieux, G., Bertozzi, C., Trends in Biotechnology, 1998, 16, 506-513). For example, when the article surface (and/or linkers grafted to its surface) displays a halo acetal, a peptide RE may be attached through a sulfhydryl moiety. A sulfhydryl moiety in the RE structure can be accomplished through inclusion of a cysteine residue.

Coupling is also possible between a primary amine on the article or the linker terminus and a carboxylic acid on the RE. A carboxylate in the peptide structure can be found either on its terminal amino acid, for linear peptides, or through the inclusion of aspartic or glutamic acid residues. The opposite configuration, where the carboxylic acid is on the article and a primary amine belongs to the peptide, is also easily accessible. Many polymerizable building blocks contain acidic moieties, which are accessible at the surface of the beads after their polymerization. As for poly(amino acid)-based REs, a primary amine function can be found either at its N-terminus (if it is linear) and/or via introduction of a lysine residue.

Another example of reactive chemical pairs consists of the coupling of a sulfhydryl with a halo acetal or maleimide moiety. The maleimide function can be easily introduced, either on a peptide, a linker, or the surface of the articles, by reacting other common functionalities (such as carboxylic acids, amines, thiols or alcohols) with linkers through methods known to one of skill in the art, such as described for example by G. T. Hermanson in Bioconjugate Techniques, Academic Press Ed., 1996. In a preferred embodiment, the inclusion of CiBA, or other disulfide-containing building blocks, in the scaffold facilitates the attachment of REs through thiol reactive moieties. After scaffold formation, reduction of the disulfide linkage in CiBA produces free thiols. Linker molecules containing groups that are reactive with thiol, such as bromoacetamide or maleimide, are added to the reduced therapeutic agent-containing article to attach the linker to the article scaffold. REs are then added, which react with the free terminus of the linker molecules to give RE-functionalized articles. Alternatively, the RE may be attached to one end of the linker molecule prior to attachment of the linker molecule to the reduced article.

Peptides can also be coupled to the article and/or the tether with a reaction between an amino-oxy function and an aldehyde or ketone moiety. The amino-oxy moiety (either on the articles or in the peptide) can be introduced, starting from other common functionalities (such as amines for example), by a series of transformations known to those skilled in the art. In the same way, aldehyde- or ketone-containing articles and aldehyde-containing peptides are readily synthesized by known methods.

The resulting RE-functionalized, drug-containing articles may be used immediately, may be stored as a liquid solution, or may be lyophilized for long-term storage.

The REs may be any small or large molecular structure that provides the desired binding interaction(s) with the cell surface receptors of the targeted molecule. The number of recognition element moieties per article can range from 2 to about 1000, preferably from 2 to 500, and most preferably from 2 to 100. The articles may optionally further be comprised of more than one type of RE. As used herein, a RE “type” is defined as a specific molecular structure.

REs preferably are comprised of peptides. Peptides used as REs according to this invention will generally possess dissociation constants between 10⁻⁴ and 10⁻⁹ M or lower. Such REs may be comprised of known peptide ligands. For instance, Phoenix Peptides' peptide ligand-receptor library (http://www.phoenixpeptide.com/Peptidelibrarylist.htm) contains thousands of known peptide ligands to receptors of potential therapeutic value. The peptides may be natural peptides such as, for example, lactams, dalargin and other enkaphalins, endorphins, angiotensin II, gonadotropin releasing hormone, melanocyte-stimulating hormone, thrombin receptor fragment, myelin, and antigenic peptides. Peptide building blocks useful in this invention may be discovered via high throughput screening of peptide libraries (e.g. phage display libraries or libraries of linear sequences displayed on beads) to a protein of interest. Such screening methods are known in the art (for example, see C. F. Barbas, D. R. Burton, J. K. Scott, G. J. Silverman, Phage Display, 2001, Cold Spring Harbor Laboratory Press, Cold Spring Harbor, N.Y.). The high affinity peptides may be comprised of naturally occuring amino acids, modified amino acids or completely synthetic amino acids. The length of the recognition portion of the peptide can vary from about 3 to about 100 amino acids. Preferably, the recognition portion of the peptide ranges from about 3 to about 15 amino acids, and more preferably from 3 to 10 amino acids. Shorter sequences are preferred because peptides of less than 15 amino acids may be less immunogenic compared to longer peptide sequences. Small peptides have the additional advantage that their libraries can be rapidly screened. Also, they may be more easily synthesized using solid-state techniques.

REs may comprise a variety of other molecular structures, including antibodies, antibody fragments, lectins, nucleic acids, and other receptor ligands. Humanized or fully human antibodies, and humanized or fully human antibody fragments are preferred for use in the present invention.

Additionally, it will be possible to design other non-protein compounds to be employed as the binding moiety, using techniques known to those working in the area of drug design. Such methods include, but are not limited to, self-consistent field (SCF) analysis, configuration interaction (CI) analysis, and normal mode dynamics computer programs, all of which are well described in the scientific literature. See, Rein et al., Computer-Assisted Modeling of Receptor-Ligand Interactions, Alan Liss, New York (1989). Preparation of non-protein compounds and moieties will depend on their structure and other characteristics and may normally be achieved by standard chemical synthesis techniques. See, for example, Methods in Carbohydrate Chemistry, Vols. I-VII; Analysis and Preparation of Sugars, Whistler et al., Eds., Academic Press, Inc., Orlando (1962), the disclosures of which are incorporated herein by reference.

The use of multiple RE molecules of the same molecular structure or of different molecular structure to make up the article can increase the avidity of the article. As used in the present invention, “high affinity” means a binding of a single RE to a single target molecule with a binding constant stronger than 10⁻⁴ M, while “avidity” means the binding of two or more such RE units to two or more target molecules on a cell or molecular complex.

The REs can target a multitude of disease-associated biomolecules. Tumor-associated targets include erbB1 (for example using the growth factor EGF, or using peptides comprised of the amino acid sequence YCPIWKFPDEECY, or other sequences found in Greene, et.al., J. Biol. Chem., 2002, 277(31), 28330-28339 as REs), erbB2 (for example using peptides comprised of the amino acid sequence CdFCDGFdYACYMDV, where dF and dY representing the D isomer of the amino acid residues) or other sequences delineated in Murali, J. Med. Chem., 2001, 44, 2565-2574 as REs), erbB3, erbB4, CMET, CEA (for example using peptides disclosed in PCT WO 01/74849 as REs), and EphA2. Vascular targets associated with multiple pathologies, including cancer, include VEGFR-1, VEGFR-2 (for example, using peptides comprised of the amino acid sequence ATWLPPR, as described in Demangel, et.al., EMBO J., 2000, 19(7), 1525-1533), integrins, including integrin αvβ3, and integrin αvβ1, and to extracellular proteins such as fibrin (which may be targeted using peptides comprised of amino acid sequences disclosed in PCT Publication WO 02/055544).

Article Attributes: As practiced in the invention, the articles have several attributes that make them excellent therapeutic candidates.

The articles can be administered by injection (subcutaneous, intravenous, intramuscular, intradermal, intraperitoneal, intracerebral, or parenteral), with intraveneous injection being a preferred route. The articles may also be suitable for nasal, pulmonary, vaginal, ocular delivery and oral administration. The articles may be suspended in a pharmaceutically acceptable carrier for administration.

Reagents and starting materials in some embodiments can be obtained commercially from chemical distributors such as Sigma-Aldrich (St Louise, Mo. and Milwaukee, Wis.), Kodak (Rochester, N.Y.), Fisher (Pittsburgh, Pa.), Pierce Chemical Company (Rockford, Ill.), Carbomer Inc. (Westborough, Mass.), Radcure (Smyrna, Ga.), and Polysciences (Niles, Ill.). PEG compounds may be purchased through NOF America Corporation (White Plains, N.Y.), and Nektar (Birmingham, Ala.). Peptides to be used as REs can be purchased from many sources, one being Bachem (King of Prussia, Pa.). Proteins may be obtained from sources such as Calbiochem (San Diego, Calif.).

The following non-limiting examples are provided to further describe how the invention may be practiced.

### EXAMPLES

### Example 1

### IMMA (Inulin Multi-Methacrylate) Synthesis

Inulin (4 g) was weighed into a 1 neck round bottom flask. A Teflon-coated stir bar was added and the flask was sealed with a septa. Anhydrous pyridine (approximately 20 mL for each grams of inulin) was then transferred into the flask, keeping the entire system under a blanket of nitrogen. The mixture was stirred until the inulin dissolved, then 1 mL of methacrylic anhydride was added dropwise, using a syringe. The reaction was allowed to continue for 16 hours. After that interval, enough pyridine was removed under vacuum to make a viscous liquid. Toluene (approximately 40 mL) was then added with vigorous mixing to precipitate the crude product. The liquid was then decanted from the precipitate. The solid was dissolved with water, producing a viscous, but free flowing, syrup, which was then precipitated with 2-propanol (approximately 200 mL). This process of water dissolution followed by precipitation was repeated twice more, after which the product was dried under vacuum. The product was then redissolved in 50 mL of water and filtered through a filter paper to remove accumulated dust and other insolubles, and the product was then lyophilized to give inulin multi-methacrylate (IMMA). IMMA identity (degree of methacrylate functionalization) was confirmed by NMR analysis.

### Example 2

### Preparation of Dextran Multi(Methacrylate) (DMMA)

Dextran (MW=5000) was dissolved in 100 mL dry DMSO with the aid of a stir bar. On complete dissolution, anhydrous pyridine was added (20 mL). Over the course of 0.5 hour, methacrylic anhydride (3.27 mL) was added dropwise, and the reaction was allowed to stir overnight. The following day, addition of toluene (150 mL) precipitated the product, and the reaction solvent/toluene mixture was decanted off of the solid. The product was dissolved in the smallest amount of water necessary to make a syrup, and added to rapidly stirred isopropanol (about 150 mL). The liquid was decanted off of the solid, and the dissolution/precipitation cycle was repeated twice more. The solid was dried under vacuum to remove trace organic solvents, dissolved in 75 mL water, and the resultant solution was filtered. The product was isolated by lyophilization and was characterized by ¹H NMR.

### Example 3

### CiBA Synthesis

Sodium hydroxide, (2.0 g) was dissolved in 70 mL of dry methanol. L-cystine (2.73 g) was added to the methanolic NaOH solution, and the round bottom flask containing the mixture was immersed in an ice water bath to maintain the reaction vessel at 0° C. Acryloyl chloride (2.22 mL) was then added to the methanolic cystine solution dropwise.

The reaction was covered and stirred for 1 hour at RT, after which the reaction solution was centrifuged and the liquid phase was decanted off into rapidly stirred ethyl acetate (120 mL). The resulting suspended solids were isolated by centrifugation and were dried under vacuum. The identity of the isolated material was confirmed by ¹H NMR as N,N′-cystinebisacrylamide (CiBA).

### Example 4

### MalAc (Malonate-Acrylamide) Synthesis

Diethylaminomalonate hydrochloride (5 g) is weighed into a 1 neck round bottom flask. Add a Teflon-coated stir bar and seal the flask using a septa. Anhydrous dichloromethane (approximately 10 mL for each gram of diethylaminomalonate) is then transferred into the flask, keeping the entire system under a blanket of nitrogen. The mixture is stirred, 3.62 mL triethylamine is added (1.1 eq.) with a syringe, followed by a dropwise addition of 2.12 ml of acryloyl chloride (1.1 eq.) with a syringe. The reaction is allowed to continue for 2 hours. The reaction mixture is extracted three times with water to remove the unreacted products [triethylamine and acrylic acid]. The organic phase is dried under vacuum and resuspended in water containing resin with strong acidic residues. The aqueous resin suspension is put on a rotary shaker and the deprotection reaction is allowed to proceed overnight. The reaction mixture is then filtered and lyophilized to yield malonate acrylamide.

### Example 5

### PEG-1500 dBA Synthesis

Poly(ethylene glycol) (average molecular weight=1500, 15.36 g) was dissolved in 75 mL of dry chloroform contained in a round bottom flask. A stir bar was added to aid in the dissolution process and to maintain reaction homogeneity. Bromoacetyl chloride (4.00 mL) was added, an air-cooled reflux condenser was attached to the round bottom flask, and the reaction mixture was heated at reflux under a nitrogen purge vented to the atmosphere (to remove HCl gas generated during the reaction). After 4 hours, more bromoacetyl chloride (1.0 mL) was added and the reaction mixture was heated for an additional 5 hours. The reaction mixture was cooled and stirred gently overnight. The following day, the solvent and excess reagent was removed under vacuum, and the residue was dissolved in saturated sodium bicarbonate. The water solution was extracted with chloroform (4 times 50 mL). The organic extractions were combined, dried over magnesium sulfate, and were filtered. Removal of the solvent under vacuum left PEG-1500 dBA as an off white solid. The identity of the product was confirmed by ¹H NMR.

### Example 6

### Preparation of Heterobifunctional PEG400, Bromoacetate and Carboxylic Acid

Poly(ethylene glycol) dibromoacetate (molecular weight average 400, PEG-400 dBA) (4.0 g) was dissolved in 150 mL pH=8, phosphate buffer (0.15 M) containing 100 mL THF. To this solution, 3-mercaptopropionic acid (0.17 g) in water (10 mL) was added with rapid mixing provided by a stir bar. The pH was adjusted to 8 again by the addition of a 1.0 M NaOH solution. Sixteen hours after the addition, the volume of the reaction was reduced under vacuum to 50 mL. The volume was increased to 150 mL by adding 100 mL pH=8.0 phosphate buffer (0.10 M) and the water solution was extracted with chloroform (2 times 50 mL) to remove unreacted PEG starting material. The pH of the solution was adjusted to 2 by adding 1.0 M HCl, and the solution was extracted again with chloroform (3 times 50 mL). The combined extracts of the pH=2.0 solution were dried with sodium sulfate and filtered. Removal of the solvent under vacuum yielded the target compound.

### Example 7

### Preparation of Heterobifunctional PEG-200, Thiol and Carboxylic Acid

Poly(ethylene glycol) dithiol (molecular weight average 200) (1.53 g) was dissolved in 150 mL pH=8, phosphate buffer (0.15 M) containing 100 mL THF. To this solution, bromoacetic acid (0.27 g) in water (10 mL) was added with rapid mixing provided by a stir bar. The pH was adjusted to 8 again by the addition of a 1.0 M NaOH solution. Sixteen hours after the addition, the solvents were removed under vacuum, yielding a viscous residue to which 100 mL of pH=8.0 phosphate buffer was added (0.050 M). The water solution was extracted with chloroform (2 times 50 mL) to remove unreacted PEG starting material. The pH of the solution was adjusted to 2 by addition of 1.0 M HCl, and the solution was extracted again with chloroform (3 times 50 mL). The combined extracts of the pH=2.0 solution was dried with sodium sulfate and filtered. Removal of the solvent under vacuum yielded the target compound.

### Example 8

### IMMA-CiBA-NaA Scaffold Formation Via Free Radical Polymerization

An aqueous phase was prepared by combining 83 wt % buffer, 14 wt % IMMA, 2 wt % CiBA, 1 wt % sodium acrylate, and Eosin Y (the photoinitiator represents from 0.001 to 0.1 wt % of the monomers mass). An oil+surfactant phase was prepared by mixing 7.3 wt % Igepal CO-210, 9.4 wt % Igepal CO-720, and 83.3 wt % cyclohexane. Three grams (3 g) of the aqueous phase was added to 30 g of the oil+surfactant phase with vigorous stirring, resulting in the formation of a reverse microemulsion. The reverse microemulsion contains surfactant-stabilized nano-droplets of aqueous phase dispersed in a continuous phase of cyclohexane. The reverse microemulsion was transferred to a 100 mL Schlenk tube and was degassed by briefly pulling a vacuum on the chilled mixture. The contents of the Schienk tube were stirred and irradiated with a UV light source for one hour to polymerize the building blocks. Once the polymerization was complete, the nanoarticles were precipitated by adding pure ethanol directly to the solution and were isolated from the reaction mixture by centrifugation. The nanoarticle-containing pellet was resuspended in deionized water. Residual surfactants and solvents were removed by standard techniques (solid phase extraction). At this point, the aqueous solution of nanoarticles were filtered and the product was isolated as a solid product after lyophilization.

### Example 9

### IMMA-CIBA-DAA Scaffold Formation Through Free Radical Polymerization

An aqueous phase was prepared by combining 83 wt % water, 14 wt % IMMA, 2 wt % CiBA and 1 wt % diacetone acrylamide (DM). An oil+surfactant phase was prepared by mixing Igepal CO-210, Igepal CO-720 and cyclohexane in a weight ratio of 1.0:1.3:9.0. Three grams (3 g) of the aqueous phase were mixed with 30 g of the oil+surfactant phase, resulting in the formation of a reverse microemulsion. The reverse microemulsion contained surfactant-stabilized nano-droplets of aqueous phase dispersed in a continuous phase of cyclohexane. To the reverse microemulsion was added an aqueous solution containing Eosin Y, where the photoinitiator represented from 0.001 to 0.1 wt % of the monomers mass. The reverse microemulsion was degassed with freeze-thawing cycles under vacuum, with N₂ gas backfill between cycles. The contents were stirred and irradiated with a UV or visible light source of at least 100 W for 20 min to two hours to polymerize the building blocks. Once the polymerization was completed, the nanoarticles were precipitated by adding 9 mL of pure ethanol directly to the solution. The nanoarticle-containing pellets were resuspended in water. Residual surfactants and solvents were removed by standard techniques (dialysis, chromatography, etc.). At this point, the aqueous solution of nanoarticles may be lyophilized, if desired.

### Example 10

### IMMA-CiBA-APMA Scaffold Formation Via Free Radical Polymerization

An aqueous phase was prepared by combining 83 wt % buffer, 14 wt % IMMA, 2 wt % CiBA, 1 wt % N-(3-aminopropyl)methacrylamide hydrochloride (APMA), and Eosin Y (the photoinitiator represents from 0.001 to 0.1 wt % of the monomers mass). An oil+surfactant phase was prepared by mixing 7.3 wt % Igepal CO-210, 9.4 wt % Igepal CO-720, and 83.3 wt % cyclohexane. Three grams (3 g) of the aqueous phase was added to 30 g of the oil+surfactant phase with vigorous stirring, resulting in the formation of a reverse microemulsion. The reverse microemulsion contains surfactant-stabilized nano-droplets of aqueous phase dispersed in a continuous phase of cyclohexane. The reverse microemulsion was transferred to a 100 mL Schlenk tube and was degassed by briefly pulling a vacuum on the chilled mixture. The contents of the Schlenk tube were stirred and irradiated with a UV light source for one hour to polymerize the building blocks. Once the polymerization was complete, the nanoarticles were precipitated by adding pure ethanol directly to the solution and were isolated from the reaction mixture by centrifugation. The nanoarticle-containing pellet was resuspended in deionized water. Residual surfactants and solvents were removed by standard techniques (solid phase extraction). At this point, the aqueous solution of nanoarticles were filtered and the product was isolated as a solid product after lyophilization.

### Example 11

### IMMA-MalAc Scaffold Formation Via Free Radical Polymerization

An aqueous phase was prepared by combining 80 wt % buffer, 14 wt % IMMA, 6 wt % malonate acrylamide (MalAc) and Eosin Y (the photoinitiator represents from 0.001 to 0.1 wt % of the monomers mass). An oil+surfactant phase was prepared by mixing 7.3 wt % Igepal CO-210, 9.4 wt % Igepal CO-720, and 83.3 wt % cyclohexane. Three grams (3 g) of the aqueous phase was added to 30 g of the oil+surfactant phase with vigorous stirring, resulting in the formation of a reverse microemulsion. The reverse microemulsion contains surfactant-stabilized nano-droplets of aqueous phase dispersed in a continuous phase of cyclohexane. The reverse microemulsion was transferred to a 100 mL Schlenk tube and was degassed by briefly pulling a vacuum on the chilled mixture. The contents of the Schlenk tube were stirred and irradiated with a UV light source for one hour to polymerize the building blocks. Once the polymerization was complete, the nanoarticles were precipitated by adding pure ethanol directly to the solution and were isolated from the reaction mixture by centrifugation. The nanoarticle-containing pellet was resuspended in deionized water. Residual surfactants and solvents were removed by standard techniques (solid phase extraction). At this point, the aqueous solution of nanoarticles were filtered and the product was isolated as a lyophilized powder.

### Example 12

### Scaffold Containing Short N-Methacryloylated Peptides

An aqueous phase was prepared by combining 82 wt % buffer, 14 wt % IMMA, 2 wt % CIBA and 2 wt % N-methacryloylated Gly-Gly peptide (made according to U.S. Pat. No. 5,037,883) with Eosin Y (the photoinitiator represents from 0.001 to 0.1 wt % of the monomers mass). An oil+surfactant phase was prepared by mixing 7.3 wt % Igepal CO-210, 9.4 wt % Igepal CO-720, and 83.3 wt % cyclohexane. Three grams (3 g) of the aqueous phase was added to 30 g of the oil+surfactant phase with vigorous stirring, resulting in the formation of a reverse microemulsion. The reverse microemulsion contains surfactant-stabilized nano-droplets of aqueous phase dispersed in a continuous phase of cyclohexane. The reverse microemulsion was transferred to a 100 mL Schlenk tube and was degassed by briefly pulling a vacuum on the chilled mixture. The contents of the Schlenk tube were stirred and irradiated with a UV light source for one hour to polymerize the building blocks. Once the polymerization was complete, the nanoarticles were precipitated by adding pure ethanol directly to the solution and were isolated from the reaction mixture by centrifugation. The nanoarticle-containing pellet was resuspended in deionized water. Residual surfactants and solvents were removed by standard techniques (solid phase extraction). At this point, the aqueous solution of nanoarticles were filtered and the product was isolated as a lyophilized powder.

### Example 13

### Scaffold Formation Via Reaction of Oxidized Inulin with a Bis Amino Oxy Compound

An aqueous phase was prepared by mixing together 85 wt % water and 15 wt % inulin. An oil+surfactant phase was prepared by mixing Igepal CO-210, Igepal CO-720 and cyclohexane in a weight ratio of 1.0:1.3:9.0. Three grams (3 g) of the aqueous phase were mixed with 40 g of the oil+surfactant phase, resulting in the formation of a reverse microemulsion. One gram of a sodium periodate solution in water was added to the mixture. The in-situ oxidation was allowed to proceed for ca. 10 minutes. A concentrated solution (of at least 1 g/mL) of bis[(2-amino-oxy)ethylamido]-(1,3-)propane in an aqueous methanol mixture (50/50 volume) was then added to the microemulsion and allowed to react overnight. The resulting nanoarticles obtained by the crosslinking of the bis[(2-amino-oxy)ethylamido]-(1,3)propane with the aldehydes functionalities of oxidized inulin were isolated by precipitation in presence of ethanol and centrifugation and purified from the excess of unreacted reagents by dialysis.

### Example 14

### Scaffold Formation Via Reaction of Oxidized Dextran with Carbo-Hydrazide

An aqueous phase was prepared by combining 90 wt % water and 10 wt % dextran. An oil+surfactant phase was prepared by mixing Igepal CO-210, Igepal CO-720 and cyclohexane in a weight ratio of 1.0:1.3:9.0. Three grams (3 g) of the aqueous phase were mixed with 40 g of the oil+surfactant phase, resulting in the formation of a reverse microemulsion. One gram of a sodium periodate solution in water was added to the mixture, so that there was a maximum of one periodate equivalent per glucose monomer unit (from the dextran). The in-situ oxidation proceeded for 5 to 20 minutes. A concentrated solution (of at least 1 g/mL) of carbohydrazide in buffer (50 to 250 mM, pH 8 to 9) was then added to the microemulsion and allowed to react overnight at 4° C. The resulting nanoarticles obtained by the crosslinking of carbohydrazide with the aldehydes functionalities of oxidized dextran were isolated by precipitation in the presence of ethanol and centrifugation, and purified from the excess of unreacted reagents by dialysis.

### Example 15

### Scaffold Formation Via Reaction of Oxidized Inulin with a Bis-hydrazine Oxy Compound

An aqueous phase was prepared by mixing together 85 wt % water and 15 wt % inulin. Two grams (2 g) of this aqueous phase were mixed with 30 g of an oil+surfactant phase composed of 8.5 wt % Igepal CO-520 in cyclohexane. One gram of a sodium periodate solution in water was added to the mixture, so that there was a maximum of one periodate equivalent per glucose monomer unit (from the inulin). The in-situ oxidation was allowed to proceed for ca. 10 minutes. A concentrated solution of succinic dihydrazine in an aqueous methanol mixture (50/50 volume) was added to the microemulsion and allowed to react for three hours, crosslinking the amino-oxy moieties with the aldehydes functions. The resulting nanoarticles were purified by precipitation and resuspended in aqueous solution in the presence of an excess of succinic dihydrazine. The reaction of succinic dihydrazine (with remaining aldehydes on the nanoarticles) was allowed to proceed for 12 hours.

### Example 16

### Doxorubicin (Dox) Attachment Via Amide Linkage

Doxorubicin (120 mg) was completely dissolved in 15.0 mL water, and 15.0 mL of HEPES buffer, pH 7.5 (0.20 M) was added. To a solution of nanoarticles of Example 8 (1.0 g), dissolved in 10.0 mL HEPES buffer, pH 7.5 (0.20 M), was added 10.0 mL water. To this solution, 160 mg of N-hydroxysuccinimide (NHS) and 50 mg 1-(3-dimethylaminopropyl)-3-ethylcarbodiimide hydrochloride (EDC) were added in succession. Twenty minutes after the addition of EDC, 7.5 mL of the doxorubicin solution was added. A second 50 mg aliquot of EDC was added, followed by a twenty minute delay before the addition of a second quantity of doxorubicin. After another 0.5 hour reaction interval, the addition/delay cycle was repeated twice more so that the entire doxorubicin solution was added. The reaction mixture was then stirred for 24 hours. The unreacted starting materials and side products were separated from the Dox-nanoarticle product by passage through a HiPrep 26/10 desalting column, and the nanoarticles were isolated as a dry solid by lyophilization. The Dox loading of the resulting articles was approximately 6 to 8 wt %. The doxorubicin content of the nanoarticles can be varied by using smaller quantities of this material during the additions or by decreasing the total number of reaction cycles.

### Example 17

### Doxorubicin Attachment Via Hydrazone Linkage (High Loading)

1.0 gram of material from Example 9 was solubilized in 40 mL of 0.1 M ammonium formate, pH 8.5, and added to a concentrated solution of carbohydrazide in excess (100 eq. carbohydrazide to each diacetone acrylamide in the starting material). The reaction was allowed to proceed for 12 hours at 4° C. An hydrazone bond was thus formed between the carboxyhydrazide and the ketone moieties of the nanoarticle. The reaction mixture was purified of the excess carbohydrazide using dialysis or size exclusion chromatography.

200 mg of doxorubicin, diluted in 100 mL water, were added to the carbohydrazide-functionalized scaffolds in water buffered by ammonium formate. The reaction was allowed to proceed for 18 to 24 hours at 4° C. A hydrazone bond was thus formed between the ketone moieties on doxorubicin and the carbohydrazide linked to the nanoarticle. The resulting nanoarticles with covalently attached doxorubicin were then purified from unreacted doxorubicin and side products using dialysis or size exclusion chromatography.

### Example 18

### Doxorurubicin Attachment Via Hydrazone Linkage (Lower Loading)

1.0 gram of material from Example 9 was solubilized in 40 mL of 0.1 M potassium borate buffer, pH 8, and added to a concentrated solution of carbohydrazide in excess (10 eq. carbohydrazide to each diacetone acrylamide in the starting material). The reaction was allowed to proceed for 1 hour. The reaction mixture was purified of the excess carbohydrazide using dialysis or size exclusion chromatography. An hydrazone bond was thus formed between the carboxyhydrazide and the ketone moieties of the nanoarticle.

50 mg of doxorubicin, diluted in 20 mL water, were added to the carbohydrazide-functionalized nanoarticle in water buffered by ammonium formate. The reaction was allowed to proceed for one to 6 hours. An hydrazone bond was thus formed between the ketone moieties on doxorubicin and the carbohydrazide linked to the nanoarticle. The resulting nanoarticles with covalently attached doxorubicin were then purified from unreacted doxorubicin and side products using dialysis or size exclusion chromatography.

### Example 19

### Cyclosporin Attachment Via an Amide Bond

Cyclosporin is mixed with 4-benzoylbenzoic acid (BBa) at a molar ratio of 1 to 2 in benzene. The solution is purged with nitrogen gas and photolysed at a wavelength of 320 nm at room temperature (U.S. Pat. No. 5,405,785). After photolysis, benzene is evaporated in a rotary evaporator under vacuum and the dried product is dissolved in methanol. The product is isolated by preparative HPLC. Cyclosporin-BBa is then added to a solution of N-hydroxysuccinimide (NHS) and dicyclohexylcarbodiimide at a molar ratio of approximately 1 to 1.2 to 1 in methanol. The reaction is allowed to run overnight at room temperature. Activated ester formation can be detected with a neutral Fe-hydroxamate test. The cyclosporin-BBa-NHS can be coupled to a nanoarticle scaffold comprised of amine moieties (the fabrication of which is described in Example 13) using EDC coupling.

### Example 20

### Calicheamicin Attachment Via a Hydrazone Bond

The synthesis of the calicheamicin derivative, N-acetyl-gamma calicheamicin dimethyl hydrazide has previously been reported (Upeslacis J., et al., Cancer Res., 1993, 53, 3336-3342). 1.0 gram of IMMA-CiBA-DAA scaffold nanoarticle is solubilized in 40 mL of 0.1 M ammonium formate, pH 8.5 and added to a concentrated solution of N-acetyl-gamma calicheamicin dimethyl hydrazide in excess (100 eq. N-acetyl-gamma calicheamicin dimethyl hydrazide to each diacetone acrylamide in the starting material). The reaction is allowed to proceed for 12 hours at 4° C. A hydrazone bond is thus formed between the N-acetyl-gamma calicheamicin dimethyl hydrazide and ketone moieties of the nanoarticle. The reaction mixture is purified of excess N-acetyl-gamma calicheamicin dimethyl hydrazide using a 26/10 desalting column.

### Example 21

### Dexamethasone Attachment

Dexamethasone conjugation to the nanoarticle is possible via a glycine-functionalized linker. This linker compound is prepared through the use of solid phase synthetic techniques via glycine-functionalized 2-CITrityl resins (2002/2003 NovaBiochem Catalog, page 2.16-2.17, CalBiochem-NovaBiochem Corp., San Diego, USA). The acid moiety of the glycine linker is reacted with the 21 carbon hydroxyl group of dexamethasone using a carbodiimide. An excess of dexamethasone is employed to react all acid groups of the glycine linker. The resulting compound is cleaved at the site of the amine using 1% trifluoroacetic acid (TFA). This produces a free amine which can be conjugated with acid (NaA) functionalized nanoarticles. The product is purified by use of a HiPrep 26/10 desalting column/size-exclusion chromatography. The ester linkage will then be cleaved in vivo, releasing the dexamethasone.

### Example 22

### Gemcitabine Incorporation

Dissolve nanoarticles (1.0 g) of Example 9 (and preferably containing DAA or oxidized sugars) in 10.0 mL PBS buffer, pH 7.2 (0.2 M). Add 15.5 mg of gemcitabine to the nanoarticle solution (at 35° C.). React for 1 hour before isolating nanoarticles via size exclusion chromatography. The aqueous solution of nanoarticles can be lyophilized at this point.

### Example 23

### Methotrexate Attachment

Methotrexate (100 mg, 0.22 mmol) is dissolved in dimethyl sulfoxide (DMSO) (10 mL) with addition of boc-protected ethanolamine (100 mg, 0.44 mmol), dicyclohexylcarbodiimide (DCC) (91 mg, 0.44 mmol) and 4-dimethylaminopyridine (DMAP) (36 mg, 0.293 mmol) at 0° C. The reaction mixture is left overnight at room temperature. The N-protected amino ester methotrexate is washed with 0.1 N HCl, dried and evaporated under reduced pressure, yielding the solid product. The boc-protected group is removed by dissolving the product in 50:50 mixture of methylene chloride and TFA and stirring for 3 hrs at room temperature. On evaporating the solvent, the deprotected amino-ester derivative is obtained (Minko T., et al., Cancer Chemother. Pharmacol., 2002, 50, 143-150).

The dried powder of the amino-ester derivative (100 mg) is completely dissolved in 2 mL DMSO. To a solution of nanoarticles of Example 8 (1.0 g), dissolved in 10.0 mL HEPES buffer, pH 7.5 (0.20 M), is added 10.0 mL water. To this solution, the methotrexate solution is added followed by the addition of 160 mg of N-hydroxysuccinimide (NHS) and 50 mg 1-(3-dimethylaminopropyl)-3-ethylcarbodiimide hydrochloride (EDC) in succession. After 30 minutes, a second 50 mg aliquot of EDC is added. After another 0.5 hour reaction interval, the addition/delay cycle is repeated twice more. The reaction mixture is then stirred for 24 hours. The unreacted starting materials and side products are separated from the methotrexate-nanoarticle product by passage through a HiPrep 26/10 desalting column, and the nanoarticles are isolated as a dry solid by lyophilization.

### Example 24

### Salicylic Acid Attachment

400 mg of succinic anhydride is reacted with 2.0 g IMMA (prepared according to Example 1) in pyridine to a level of approximately 1 carboxylic acid moieties per 3 saccharide repeat groups of inulin and isolated according to the method described in Example 1.1.0 g of this acid modified IMMA (AM-IMMA) is then coupled to 300 mg of salicylic acid in DMF using 1,3-dicyclohexylcarbodiimide(DCC, 500 mg). In this procedure, the AM-IMMA is dissolved in anhydrous DMF (50 mL) and the DCC is added. After 4 hours, the salicylic acid is added and the reaction is stirred overnight. The following day, the reaction is filtered, and the DMF is removed under vacuum. The crude material is dissolved in water (50 mL), filtered again, and the product is isolated after lyophilization. This salicylic acid-modified SAM-IMMA is then used in the formulation procedure described in Example 8, instead of the unmodified IMMA, with the synthesis and isolation procedures remaining unchanged, to produce SAM-IMMA/CiBA/sodium acrylate nanoarticle scaffolds.

### Example 25

### Camptothectin Attachment

20-O-peptidyl-camptothecin (120 mg) (prepared according to the procedure in Farao M., et al. (Molecular Cancer Therapeutics, 2003, 2, 29-40) was dissolved in 15 mL DMSO, and 15.0 mL of HEPES buffer, pH 7.5 (0.20 M) was added. To a solution of nanoarticles (1.0 g) made according to Example 8 (acid-containing nanoarticles), dissolved in 10.0 mL HEPES buffer, pH 7.5 (0.20 M), was added 10.0 mL water. To this solution, 160 mg of N-hydroxysuccinimide (NHS) and 50 mg 1-(3-dimethylaminopropyl)-3-ethylcarbodiimide hydrochloride (EDC) were added in succession. Twenty minutes after the addition of EDC, 7.5 mL of the 20-O-peptidyl-camptothecin solution was added. A second 50 mg aliquot of EDC was added, followed by a twenty-minute delay before the addition of a second quantity of 20-O-peptidyl-camptothecin. After another 0.5 hour reaction interval, the addition/delay cycle was repeated twice more so that the entire 20-O-peptidyl-camptothecin solution was added. The reaction mixture was then stirred for 24 hours. The unreacted starting materials and side products were separated from the 20-O-peptidyl-camptothecin nanoparticle product by passage through HiPrep 26/10 desalting column, and the nanoarticles were isolated as a dry solid by lyophilization.

### Example 26

### Cis-diamino-platinum N,O Complexed with Malonate-Containing Nanoarticles

To a solution of 1.0 g nanoarticles as described in Example 11 (composed of 80 wt % buffer, 14 wt % IMMA, 6 wt % MalAc, and Eosin Y), in 0.1M sodium nitrate pH 7.4 is added 26 mL of a 50 mM solution of cis-diaminediaquaplatinum dinitrate in sodium nitrate. The reaction is allowed to proceed for 12 hours at room temperature. The use of nitrate favors the ligand exchange. The O,O-platinum-malonate complex initially formed will rearrange itself into the more stable O,N-malonate complex (the transformation can be followed by Pt NMR). The unreacted platinum derivatives are then separated from the platinum-complexed-nanoarticle product by passage through a HiPrep 26/10 desalting column. The nanoarticles are isolated as a dry solid by lyophilization.

### Example 27

### Cis-Diamino-Platinum N,O Complexed with the Amide and Acid Moieties of CIBA

To a solution of 1.0 g nanoarticles composed of 80 wt % buffer, 14 wt % IMMA, 6 wt % CiBA, and Eosin Y, in 0.1 M sodium nitrate pH 7.4 was added 18 mL of a 100 mM solution of cis-diaminediaquaplatinum dinitrate in sodium nitrate. The use of nitrate favors the ligands exchange. The reaction was allowed to proceed overnight at room temperature. The unreacted platinum derivatives as well as those too loosely complexed were separated from the platinum-complexed-nanoarticle product by passage through a HiPrep 26/10 desalting column. The nanoarticles were isolated as a dry solid by lyophilization.

### Example 28

### Targeted Cis-Platin Nanoparticles [Loading Post Targeting]

Nanoarticles (composed of 82 wt % buffer, 14 wt % IMMA, 2 wt % CIBA and 2 wt % N-Methacryloylated Gly-Gly peptide, made according to U.S. Pat. No. 5,037,883 with Eosin Y) made according to Example 12 were reduced following the procedure described in Example 30 (DTT reduction), functionalized with PEG dBA (Example 30), and targeted with an ErbB-2 ligand (bromoacetamide functionalized cyclic F[CDGFYAC]YMDV) as described in Example 37.

To a solution of 1.0 g of these nanoarticles in 0.1M sodium nitrate pH 7.4 was added 18 mL of a 100 mM solution of cis-diaminediaquaplatinum dinitrate in sodium nitrate. The reaction was allowed to proceed overnight at room temperature. The unreacted platinum derivatives as well as those loosely associated were separated from the platinum-complexed-nanoarticle product by passage through a HiPrep 26/10 desalting column. The nanoarticles were isolated as a dry solid by lyophilization.

### Example 29

### 5-Fluorouracil (5FU) Attachment

The chloro-methyl ester of Boc-protected glycine is attached to 5FU following the method of Taylor et. al. (Taylor H. E., Sloan K. B. Journal of Pharmaceutical Sciences, 1998, 87, 15). After deprotection with trifluoroacetic acid (TFA), and removal of excess TFA under vacuum, the amine-functionalized 5FU (made from 198 mg of the Boc-protected starting material) is used immediately for attachment to carboxylic acid containing nanoarticles. This material is completely dissolved in 15.0 mL water, and 15.0 mL of HEPES buffer, pH 7.5 (0.20 M) is added. To a solution of nanoarticles of Example 8 (1.0 g), dissolved in 10.0 mL HEPES buffer, pH 7.5 (0.20 M), 10.0 mL of water is added. To this solution, 160 mg of N-hydroxysuccinimide (NHS) and 50 mg 1-(3-dimethylaminopropyl)-3-ethylcarbodiimide hydrochloride (EDC) is added in succession. Twenty minutes after the addition of EDC, 7.5 mL of the amine functionalized 5FU solution is added. The resulting solution is stirred gently for 0.5 hour. A second 50 mg aliquot of EDC is added, followed by a twenty minute delay before the addition of a second quantity of the derivatized 5FU. After another 0.5 hour reaction interval, the addition/delay cycle is repeated twice more so that the entire derivatized 5FU solution is added. The reaction mixture is then stirred for 24 hours. The unreacted starting materials and side products are separated from the nanoarticle product by passage through a HiPrep 26/10 desalting column and the nanoarticles are isolated as a dry solid by lyophilization.

### Example 30

### Linker Attachment

Doxorubicin-loaded articles (400 mg) described in Example 16 were dissolved in 0.5 mL phosphate buffer, pH=7.2, 0.10 M, containing 0.15 M NaCl (this buffer is frequently referred to as PBS). To that solution, dithiothreitol (DTT, 198 mg) was added, and the reduction reaction was stirred gently for 2.0 hours. The resulting articles were separated from other materials by use of a Fast Performance Liquid Chromatography (FPLC) system equipped with a HiPrep 26/10 desalting column using 100 mM phosphate buffer (pH=8.0) as the eluting solvent. PEG-1500 dBA (2.59 g) was weighed into a 50 mL vial. The freshly purified nanoarticle solution was added to the PEG-1500 dBA, and the PEG attachment reaction was stirred gently for 2 hours at room temperature under aluminum foil. The nanoarticles were then separated from the other components of the reaction mixture using the same FPLC arrangement described earlier. The pooled fractions containing nanoarticles were used in subsequent procedures, outlined in following examples, in which specific targeting elements are attached.

### Example 31

### BMPEO₄ Attachment

Doxorubicin-loaded articles (400 mg) described in Example 16 were dissolved in PBS. To that solution, dithiothreitol (DTT, 198 mg) was added, and the reduction reaction was stirred gently for 2.0 hours. The articles were separated from other materials by use of a FPLC equipped with a HiPrep 26/10 desalting column using PBS as the eluting solvent. The homobifunctional crosslinker 1,11-bis-maleimidotetraethyleneglycol (BMPEO₄, 379 mg) was added to the pooled FPLC fractions which contained the nanoarticles. The reaction was allowed to run for 2 hr at room temperature with agitation. The unreacted linker was then removed from the nanoarticles using the same FPLC described previously. These materials are used in a fashion similar to the PEG-dBA functionalized particles.

### Example 32

### erbB1 Ligand Attachment to Dox-Loaded Article

The disulfide-bridged cyclic peptide 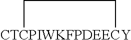
(174 mg) was added to the pooled fractions eluting off the FPLC that contained the nanoarticles described in Example 16. This reaction was stirred gently for 2 hours under aluminum foil. Separation of the nanoarticles from the other components in the reaction mixture was achieved by the use of an FPLC equipped with a HiPrep26/10 desalting column, using de-ionized water as the solvent. The nanoarticle products may be isolated by lyophilization.

### Example 33

### erbB2 Ligand Attachment to Dox-Loaded Article

The disulfide-bridged cyclic peptide 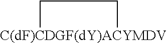
(the notation dF representing the D isomer of phenylalanine and dY representing the D isomer of tyrosine, 162 mg) was added to the pooled fractions eluting off the FPLC that contained the nanoarticles described in Example 16. This reaction was stirred gently for 2 hours under aluminum foil. Separation of the nanoarticles from the other components in the reaction mixture was achieved by the use of an FPLC equipped with a HiPrep26/10 desalting column, using de-ionized water as the solvent. The nanoarticle products may be isolated by lyophilization.

### Example 34

### VEGFR2 Ligand Attachment to Dox-Loaded Article

The peptide ATWLPPRC (120 mg) was added to the pooled fractions eluting off the FPLC that contained the nanoarticles described in Example 16. This reaction was stirred gently for 2 hours under aluminum foil. Separation of the nanoarticles from the other components in the reaction mixture was achieved by the use of an FPLC equipped with a HiPrep26/10 desalting column, using de-ionized water as the solvent. The nanoarticle products may be isolated by lyophilization.

### Example 35

### RGD Attachment to PEG dBA Functionalized Articles

Cyclic RGD peptide (HAP3C, RGDdFC, or FW 576) (62 mg) was added to a solution containing 355 mg of doxorubicin-containing nanoarticles with attached PEG₄₀₀ dBA linker tethers (prepared by functionalizing Dox-articles of Example 17 with PEG₄₀₀ dBA following the procedures of Example 30) at 10 mg/mL in PBS, pH 8. The reaction was allowed to run for 2 hours, after which 13 mg of cysteine was added. The reaction with cysteine was allowed to run for 1 hour, after which the nanoarticles were purified by FPLC with a volatile buffer, pH 7 to 9 as eluent to give doxorubicin-containing nanoarticles with RGD attached via PEG₄₀₀ dBA chains. The nanoarticles were lyophilized.

### Example 36

### erbB1 Ligand Attachment to PEG dBA Functionalized Articles

100 mg of cyclic ErB1 peptide, with a cysteine on the N terminus (CYCPIWKFPDEECY) was added to a solution containing 300 mg of doxorubicin-containing nanoarticles with attached PEG₄₀₀ dBA linker tethers (prepared by functionalizing Dox-articles of Example 17 with PEG₄₀₀ dBA following the procedures of Example 30) at 5 mg/mL in PBS, pH 8. The reaction was allowed to run for 4 hours, after which 11 mg of cysteine was added. The reaction with cysteine was allowed to run for 1 hour, after which the nanoarticles were purified by FPLC to give doxorubicin-containing nanoarticles with Erb1 attached via PEG₄₀₀ dBA chains.

### Example 37

### erbB2 Ligand Attachment to PEG dBA Functionalized Articles

50 mg of cyclic ErB1 peptide, with a cysteine on the N terminus (CdFCDGFdYACYMDV) was pre-dissolved in water and added to a solution containing 300 mg of doxorubicin-containing nanoarticles with attached PEG₄₀₀ dBA linker tethers (prepared by functionalizing Dox-articles of Example 17 with PEG₄₀₀ dBA following the procedures of Example 30) at a final concentration of 2.5 mg/mL in PBS, pH 8. The reaction was allowed to run for 4 hours, after which 11 mg of cysteine was added. The reaction was allowed to run for 2 hours, after which the nanoarticles were purified by FPLC to give doxorubicin-containing nanoarticles with ErB2 attached via PEG₄₀₀ dBA chains.

### Example 38

### VEGFR-2 Ligand Attachment to PEG dBA Functionalized Articles

A cysteine end-terminated VEGFR-2 peptide (ATWLPPRC) (100 mg) was added to a solution containing 355 mg of doxorubicin-containing nanoarticles with attached PEG₄₀₀ dBA linker tethers (prepared by functionalizing Dox-articles of Example 17 with PEG₄₀₀ dBA following the procedures of Example 30) at 10 mg/mL in PBS, pH 8. The reaction was allowed to run for 2 hours, after which 13 mg of cysteine was added. The reaction was allowed to run for 1 hour, after which the nanoarticles were purified by FPLC to give doxorubicin-containing nanoarticles with VEGFR-2 attached via PEG₄₀₀ dBA chains.

### Example 39

### Tolerability of Hydrogel Scaffold in Mice

Scaffold nanoarticles of 14 wt % IMMA and 1 wt % DAA were prepared following the procedures described in Example 9 but without the addition of CiBA.

Nine female SCID/Rag 2M mice (age 9 weeks, weight 18-22 g) were randomly grouped into 3 groups of three mice prior to study initiation. Articles were solubilized in saline solution and injected intravenously into the tail veins of the mice. Intravenous administration of nanoarticles at 100, 200, and 400 mg/kg was well tolerated with no acute or delayed signs of toxicity. Weights were monitored over a period of 16 days. During that time no weight loss was observed. Results are shown in Table 1:

TABLE 1
average weight change in mice (n = 3) in % after single intravenous
injection of nanoarticles.

| | Dose in mg/kg | | |
|---|---|---|---|
| day | 100 (SD) | 200 (SD) | 400 (SD) |
| 1 (treatment) | 0 (0) | 0 (0) | 0 (0) |
| 2 | 0.68 (0.78) | 1.28 (1.11) | −0.77 (2.38) |
| 5 | −1.19 (2.11) | −1.62 (1.97) | −0.59 (5.83) |
| 6 | 2.67 (2.77) | 3.22 (1.19) | 3.91 (4.70) |
| 7 | 2.84 (2.09) | 2.72 (3.28) | 2.21 (5.14) |
| 8 | 1.01 (2.66) | 1.75 (2.95) | 1.74 (3.61) |
| 9 | 1.85 (3.27) | 4.49 (3.08) | 20.4 (4.22) |
| 12 | 4.37 (2.11) | 7.06 (5.03) | 2.65 (1.89) |
| 14 | 0.83 (5.53) | 11.24 (5.50) | 9.90 (3.89) |
| 16 | 5.37 (4.03) | 11.59 (5.50) | 11.58 (2.20) |

### Example 40

### PK of FITC-Labeled NP Matrix

FITC-labeled nanoparticles (NP) or FITC-labeled nanoparticles conjugated with PEG-400 (NP-PEG) were made using the reverse microemulsion polymerization method described in Example 8, where the aqueous phase was comprised of 83 wt % water, 14 wt % IMMA, 1 wt % APMA, and 1 wt % NOBA. Fluorescein Isothiocyanate (FITC) was attached via linkage between the isothiocyanate of the fluorescein and the amine-containing moiety aminopropyl methacrylamide (APMA) on the nanoparticles.

Female Balb/c mice were injected intravenously with 50 mg/kg of FITC-labeled nanoparticles (NP) or FITC-labeled nanoparticles conjugated with PEG-400 (NP-PEG). The treatment groups were assessed at 15-minutes, 2-hours, 6-hours, and 24-hours. Blood and tissue samples were collected at the indicated time points. Tissue samples were snap frozen prior to further analysis. From the blood samples plasma was prepared via centrifugation at 1500 g for 10 minutes. Plasma and tissues were assayed for the presence of NP or NP-PEG via a fluorescence assay using a microplate fluorescence reader at an excitation wavelength of 485 nm and emission wavelength of 535 nm. Plasma samples were diluted 10-fold dilution with PBS.

Final assayed volumes were 100 μl per well in black-bottom 96-well plates. Tissues were extracted in 2 ml of PBS containing 0.05% Triton X-100 using a tissue homogenizer. Then tissue samples were diluted 5-fold in PBS with a final volume of 100 μl per well in black-bottom 96-well plates.

Plasma elimination of FITC-NP represented in μg of particles indicated a long half-life in the blood of 6.6 hours for both the bare (non-functionalized) nanoparticles and nanoparticles conjugated with PEG-400. The biodistribution was analyzed by measuring the fluorescence in liver, spleen, kidneys, heart, and lungs. Low accumulation in the liver and spleen was detected for both types of nanoparticles from organ extracts taken at the 4 time points over 24 hours. No nanoparticles were found in the lungs, heart, and kidneys.

TABLE 2
Clearance of FITC-labeled NP from total blood compartment during
24 hours. Total amount of injected nanoparticles per mouse
(normalized to 20 g of weight) was 1000 μg. Values in table
are in μg per tissue.

| | 0.25 h | 2 h | 6 h | 24 h |
|---|---|---|---|---|
| NP (blood) | 772 | 648 | 408 | 143 |
| NP-PEG (blood) | 826 | 678 | 416 | 167 |
| NP (liver) | 30 | 40 | 49 | 39 |
| NP-PEG (liver) | 58 | 60 | 77 | 40 |
| NP (spleen) | 2.7 | 3.8 | 5.9 | 11 |
| NP-PEG(spleen) | 5.1 | 5.8 | 8.0 | 13 |

### Example 41

### CAM Results for RGD-Dox-NPs

The chorioallantoic membranes (CAM) of 7-days old chicken embryos were incubated with each 10⁵ cells of the murine mammary carcinoma Cl-66 24 hours prior to a 3-day incubation with disks containing either 5 μg of Doxorubicin, 5 μg of Dox equivalent in nanoparticles (14/1/1 IMMA/NOBA/NaA, PEG₃₄₀₀), 5 μg of Dox equivalent in nanoparticles functionalized with cyclic-RGD (14/1/1 IMMA/NOBA/NaA, PEG₃₄₀₀-cRGD), or PBS as a control. Linker attachment to nanoparticles was made as described in Example 31.

After 3 days, CAMs were excised and photographed for analysis. Doxorubicin alone was most effective in reducing the number of blood vessels. However, its application was also most toxic since the CAMs of 6 out of 10 embryos of this group became necrotic and 3 out of 10 embryos died during treatment. Dox-NP was performing well in altering the blood vessel distribution around the disk. Dox-NP-cRGD was more effective in reducing the number of blood vessels around the disk when compared to Dox-NP.

### Example 42

### In Vivo Toxicity/Efficacy Data for RGD-Dox NPs and Dox NPs

The efficacy and toxicity of nano-article-conjugated doxorubicin (Dox-NP) was compared to doxorubicin (Dox) and a saline control. Dox-NP of composition 14/1/1 IMMA/CiBA/NaA, 5.6 w % Dox were made following procedures described in Example 16.

CL66 murine mammary carcinoma cells were grown to 80-90% confluence in supplemented media, trypsinized for 2 min at 37° C. with 1× trypsin, rinsed with Hank's Balanced Salt Solution (w/out calcium and magnesium) and resuspended at 1×10⁶ cell/ml, with 0.1 ml injected into the mammary fat pad of female Balb/C mice.

Lyophilized Dox-NP was reconstituted in sterile PBS at the appropriate concentration and stored at 2-8° C. for at least 1 hour, or overnight. Excipient Dox was diluted in PBS at the appropriate concentrations. 200 μL was injected intravenously through the tail vein of the mice on days 13, 15, and 18 following tumor challenge. Dox was injected at 225 μg/animal, while Dox-NP was injected at a nano-article level sufficient to deliver Dox at 250, 420 and 560 μg/animal.

Tumor volumes were measured superficially in two dimensions. Weight loss (or gain) and survival were recorded. A significant reduction in tumor size was observed with 250 μg/animal. The higher doses of nanoparticles containing Dox were toxic, as was the 225 μg/animal dose of Dox, although the mice injected with Dox had a slightly longer survival time. The lowest dose of Dox-NP had therapeutic activity, resulting in slower tumor growth and thus smaller tumors. At the time of death, the tumors were smaller in the 250 μg/animal Dox-NP group and their weight was lower than those of the Dox group. However, the smaller tumors the difference in survival was minimal (post tumor injection) between the 250 μg/animal Dox-NP and control mice. This suggests that Dox-NP had activity, which was reversed with continued tumor growth in the absence of additional therapy.

### Example 43

### In Vivo Toxicity/Efficacy Data for RGD-Dox NPs and Dox NPs

The efficacy and toxicity of nano-article-conjugated doxorubicin (Dox-NP and Dox-NP-cRGD)) was studied. Dox-NPs of composition 14/1/1 IMMA/CiBA/NaA, 5.6 w % Dox, PEG₄₀₀ (with and without cRGD) were made following procedures described in Example 16. PEG₄₀₀ dBA was linked following procedures described in Example 30. Tumor-bearing mice were inoculated, treated, and observed as described in Example 42 (above). Intravenous injections were given 3 times a week for one week at cumulative doses of 1200 μg Dox equivalent for Dox-NP and Dox-NP-cRGD. Only one mouse in the Dox-NP group died and all animals in the Dox-NP-cRGD group survived. Average tumor volumes increased more than 30-fold in the saline control group, versus 12-fold in the Dox-NP group, and 6-fold in the Dox-NP-cRGD group. Larger tumors in the Dox-NP-cRGD group showed significant necrosis.

TABLE 3
Changes in tumor sizes (n = 5) represented as average x-fold increase
in volumes after treatment (days post treatment initiation (tiw)).

| days | Saline | Dox-NP | Dox-NP-cRGD |
|---|---|---|---|
| 5 | 4 | 2 | 1 |
| 7 | 3 | 2 | 2 |
| 12 | 7 | 6 | 2 |
| 15 | 8 | 5 | 3 |
| 19 | 9 | 5 | 4 |
| 22 | 18 | 8 | 4 |
| 27 | 32 | 11 | 6 |

(NA = not applicable)

### Example 44

### In Vitro Cytotoxicity of Dox-Amide Linked Nanoparticles

Doxorubicin may be attached to the nanoarticle scaffold through linkages comprised of covalent bonds that are degraded very slowly or faster in the intracellular environment. For instance, amide bonds are degraded more slowly, while other linkages that are more rapidly degraded through hydrolysis such as linkages comprised of hydrazone bonds. The toxicity of Dox linked to the nanoarticles via the amide bond was tested in an in vitro toxicity assay against C-32 cells (human melanoma). Dox-nanoarticles of composition 14/2/1 IMMA/CiBA/NaA, were made following procedures described in Example 8.

In a 96-well tissue culture microtiter plate, 10,000 cells of C-32 per well in culture medium were allowed to attach for 2 hours prior to incubation with varying concentrations of Dox-amide nanoarticle, and free Dox. Cells were incubated at 37° C., 5% CO₂ in a humidified chamber for 48 hours. Cell proliferation was assayed using the calorimetric MTT kit (CT01, Chemicon, Int.). The 50% growth inhibition was determined to 0.3 μg/mL of Dox equivalent compared to 0.8 μg/mL of free Dox.

### Example 45

### In Vitro Toxicity of Dox-Hydrazone Linked Nanoparticles

The toxicity of Dox linked to the nanoarticles via the hydrazone bond was tested in an in vitro toxicity assay against C-32 cells (human melanoma). Dox-nanoarticles of composition 14/2/1 IMMA/CiBA/DAA, were made following procedures described in Example 9.

In a 96-well tissue culture microtiter plate, 10,000 cells of C-32 per well in culture medium were allowed to attach for 2 hours prior to incubation with varying concentrations of Dox-hydrazone nanoarticle, and free Dox. Cells were incubated at 37° C., 5% CO₂ in a humidified chamber for 48 hours. Cell proliferation was assayed using the calorimetric MTT kit (CT01, Chemicon, Int.). The 50% growth inhibition was determined to 0.07 μg/mL of Dox equivalent compared to 0.8 μg/mL of free Dox.

1. The compound N,N′-cystinebisacrylamide of the following formula I:

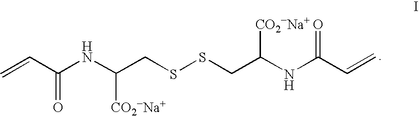

2. The compound inulin multi-methacrylate of the following formula IV, where n is from about 5 to about 50:

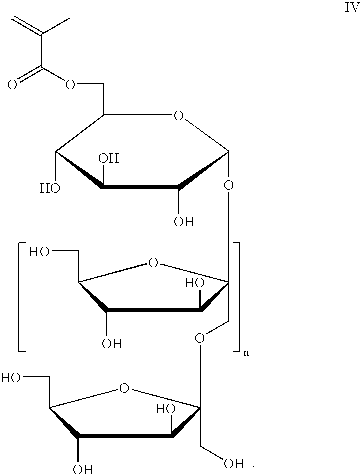

3. A hydrogel nanoarticle comprising i) a polymeric scaffold comprising crosslinked hydrophilic building blocks, and ii) a therapeutic agent covalently attached to the polymeric scaffold.

4. A hydrogel nanoarticle according to claim 3 which further comprises: iii) two or more recognition elements covalently attached to the polymeric scaffold, the recognition elements having binding affinity to biomolecular structures expressed on certain cells or in certain tissues.

5. A hydrogel nanoarticle according to claim 4 wherein at least one of the recognition elements is the amino acid sequence RGD or the growth factor EGF.

6. A hydrogel nanoarticle according to claim 3 wherein the scaffold comprises at least some degradable covalent linkages.

7. A hydrogel nanoarticle according to claim 3 wherein the hydrophilic building blocks further comprise small molecule crosslinking agents.

8. A hydrogel nanoarticle according to claim 3 wherein at least some of the hydrophilic building blocks are carbohydrate-based monomers.

9. A hydrogel nanoarticle according to claim 3 wherein at least some of the hydrophilic building blocks are inulin multi-methacrylate, N,N′-cystinebisacrylamide, diacetone acrylamide, or aminopropyl methacrylamide.

10. A hydrogel nanoarticle according to claim 3 wherein the building blocks comprise inulin multi-methacrylate, N,N′-cystinebisacrylamide, and sodium acrylate.

11. A hydrogel nanoarticle according to claim 3 wherein the building blocks comprise inulin multi-methacrylate, N,N′-cystinebisacrylamide, and diacetone acrylamide.

12. A hydrogel nanoarticle according to claim 3 which further comprises at least one polyethylene glycol molecule covalently attached to the polymeric matrix.

13. A hydrogel nanoarticle according to claim 3 wherein the therapeutic agent is a chemotherapeutic.

14. A hydrogel nanoarticle according to claim 13 wherein the therapeutic agent is doxorubicin or a doxorubicin analogue.

15. A hydrogel nanoarticle according to claim 3 wherein the therapeutic agent is attached to the scaffold through a hydrolyzable linkage.

16. A method for the controlled delivery of a therapeutic agent to the vicinity of a targeted cell or tissue type, the method comprising administering to an environment containing the targeted cell or tissue type, a hydrogel nanoarticle comprising i) a polymeric scaffold comprising crosslinked hydrophilic building blocks; ii) a therapeutic agent covalently attached to the scaffold, and iii) two or more recognition elements covalently attached to the scaffold, the recognition elements having binding affinity to biomolecules expressed on the targeted cell or in the tissue type.

17. A method for synthesizing a hydrogel recognition element-functionalized polymeric nanoarticle, the method comprising:
forming a nanoarticle polymeric scaffold through the crosslinking of hydrophilic building blocks in the dispersed aqueous phase of a reverse microemulsion, wherein at least some of the building blocks are N,N′-cystinebisacrylamide;
reducing the polymeric scaffold to produce free thiols from the disulfide linkage of the N,N′-cystinebisacrylamide;
adding linker molecules, the linker molecules containing groups that are reactive with thiol, to attach the linker to the polymeric scaffold; and
adding recognition elements, the recognition elements containing groups that are reactive with the free terminus of the linker molecules;
to give recognition element-functionalized nanoarticles.

18. A method according to claim 17 which further comprises the step of adding therapeutic agents to the nanoarticle prior to reducing the polymer scaffold, the therapeutic agent having groups that are reactive with the polymeric scaffold to covalently attach the therapeutic agents to the scaffold.

19. A method for synthesizing a hydrogel recognition element-functionalized polymeric nanoarticle, the method comprising:
forming a nanoarticle polymeric scaffold through the crosslinking of hydrophilic building blocks in the dispersed aqueous phase of a reverse microemulsion, wherein at least some of the building blocks are N,N′-cystinebisacrylamide;
reducing the polymeric scaffold to produce free thiols from the disulfide linkage of the N,N′-cystinebisacrylamide; and
adding linker molecules comprising a recognition element attached to one end of the linker molecule, the linker molecules containing groups that are reactive with thiol, to attach the recognition element-functionalized linker molecule to the polymeric scaffold;
to give recognition element-functionalized nanoarticles.

20. A method according to claim 19 which further comprises the step of adding therapeutic agents to the nanoarticle prior to reducing the polymer scaffold, the therapeutic agent having groups that are reactive with the polymeric scaffold to covalently attach the therapeutic agents to the scaffold.
